(19) United States
(12) Patent Application Publication
(10) Pub. No.: US 2003/0143648 A1
(43) Pub. Date: Jul. 31, 2003

(54) Enzyme activity profiles

(76) Inventors: Benjamin F. Cravatt, La Jolla, CA (US); Nadim Jessani, San Diego, CA (US); Yongsheng Liu, San Diego, CA (US)

Correspondence Address: GRAY CARY WARE & FRIEDENRICH LLP, 4365 EXECUTIVE DRIVE, SUITE 1100, SAN DIEGO, CA, 92121-2133, US

(21) Appl. No.: 10/304,559

(22) Filed: Nov. 25, 2002

Related U.S. Application Data
(60) Provisional application No. 60/334,426, filed on Nov. 29, 2001.

(51) Int. Cl. G01N 33/574

(52) U.S. Cl. 435/7.23

(57) ABSTRACT

Methods and compositions are provided for evaluating cellular status related to neoplasia. Affinity based probes, particularly having fluorophosphonates as a reactive functionality, are employed that react with a family of enzymes having a common catalytic activity, particularly serine/threonine hydrolases. The probe(s) are combined with the cell components and resulting conjugates are characterized, where the profile of reaction indicates cellular status. A novel serine/threonine hydrolase enzyme is provided.

### RELATED APPLICATION DATA

This application claims the benefit of priority under 35 U.S.C. 119(e) of U.S. Serial No. 60/334,426, filed Nov. 29, 2001, the entire contents of which is incorporated herein by reference.

### GOVERNMENT SUPPORT

This invention was made in part with government support under grants from the NCI of the NIH, the California Breast Cancer Research Program, ActivX Biosciences and the Skaggs Institute for Chemical Biology. The government may have certain rights in this invention.

### BACKGROUND OF THE INVENTION

1. Field of the Invention

The invention relates generally to enzyme profiling in evaluating cell status.

2. Background Information

The ability to follow the intracellular events using a variety of protocols has opened opportunities for identifying the intracellular events associated with diseased cells, e.g. hyperplasia and neoplasticity, response to environments, e.g. drugs and other treatments, and the better understanding of the cellular pathways and their interweaving in response to a number of different conditions. Popularly today, arrays are being described that allow for the dissection of the transcription pattern of a cell or the proteins present in a cell. By comparing normal cells with diseased cells or cells subjected to a standard environment as compared to a test environment, one can determine how the transcription profile or the proteomic profile has been changed.

In analyzing the proteome of a cell, there are many different categories of cellular components that one can measure: mRNA, proteins, protein locations, protein complexes, modified proteins, etc. Each of these may be varied, depending on the individual, the particular time of the measurement, response to various changes, such as eating, circadian rhythm, stage in proliferation, or other event that may have nothing to do with the status of interest, but may affect the cellular composition. Discovering which proteins have relevance to the cellular status is a significant enterprise.

Conventional proteomics approaches that rely on two-dimensional gel electrophoresis encounter difficulties analyzing membrane-associated and low abundance proteins. Additionally, most proteomics technologies are restricted to detecting changes in protein abundance and, therefore, offer only an indirect readout of dynamics in protein activity. Numerous posttranslational forms of protein regulation including those governed by protein-protein interactions remain undetected. Each of these posttranslational modifications may have a prominent effect on the status of the cell, where determining only the presence of the protein may be misleading. The large numbers of proteins present in a cell, their dynamic response to changes in the status and environment of the cell, and the changes in the proteins, makes finding correlations between portions of the proteomic profile and useful information concerning disease conditions, response to drugs and useful therapeutic regimes problematic.

Thus, a need exists for ways to interrogate the proteome of cells in a variety of situations, where useful information is forthcoming that allows for the diagnosis and treatment of diseases.

### SUMMARY OF THE INVENTION

Enzyme, exemplified by serine hydrolase, profiles are provided, where variations in the profile are related to cellular status, particularly as to neoplastic status, including identification of the origin of tumors and their stage in the progression of the tumor, and novel enzymes associated with tumors. Also, methods for analyzing neoplastic cells as to their origin, invasiveness and response to therapeutic treatment are provided. Particularly, probes reactive with the active site of enzymes present in the cells are combined with components of the cells, particularly as a lysate, and the enzyme profile determined by means of ligands present as part of the probes.

### BRIEF DESCRIPTION OF THE FIGURES

FIGS. 1A-1B. Serine hydrolase activity profiles of the secreted proteomes of cancer cell lines. A. A representative in-gel fluorescence analysis of secreted serine hydrolase activity profiles obtained from reactions between cancer cell line conditioned media and a rhodamine FP (“FP”—fluorophosphonate). Enzyme activities are identified on either side of the gel (arrowheads point to the deglycosylated form of each enzyme; see FIG. 3a for full names of proteins). Deglycosylation was accomplished by treatment of a portion of the FP-labeled proteomes with PNGaseF prior to analysis. APH* refers to acyl peptide hydrolase, an abundant cytosolic enzyme also detected in the conditioned media. B. Levels of active urokinase secreted by cancer cell lines as measured by ABPP (“ABPP”—activity-based protein profiling). c. Inhibition of urokinase activity by PA-I. Pre-treatment of each proteome with PA-I (20 μg/mL) blocked the labeling of urokinase by FP-rhodamine, but did not affect the labeling of other serine proteases (complement component 1 s and cathepsin A).

FIGS. 2A-2E. Serine hydrolase activity profiles of the membrane and soluble proteomes of cancer cell lines. Shown in A and B are representative in-gel fluorescence analyses of serine hydrolase activity profiles of the membrane (A) and soluble (B) proteomes of cancer cell lines. Enzyme activities are identified on either side of the gels (arrowheads point to the deglycosylated form of each enzyme; see FIG. 3A for full names of proteins). Asterisked proteins represent soluble hydrolases also detected in the membrane proteome. NS* refers to a non-specifically labeled protein also detected in heat-denatured control proteomes (data not shown); DG, deglycosylated. C. The activity of fatty acid amide hydrolase (FAAH) in breast cancer membranes as measured by ABPP (left graph) and FAAH substrate (right graph) assays. D. Activity levels for a novel membrane amidase KIAA1363 in melanoma (left panel) and breast carcinoma (right panel) lines (shown are data for the upper glycosylated form of this enzyme). E. The ratio of upper to lower glycosylated forms of KIAA1363 in breast carcinoma lines.

FIGS. 3A-3C. Clustering of serine hydrolase activity profiles. A. Hierarchical clustering analysis of total serine hydrolase activity profiles of cancer cell lines. B. Clustering analysis of secreted and membrane serine hydrolase profiles. C. Clustering analysis of soluble serine hydrolase activity profiles. Bars to the left of the dendrograms represent similarity scores. The intensity of blue color scales directly with the relative activity of each hydrolase among the cell lines (0-100%, where for each enzyme 100%represents the cell line with the highest activity and the rest of the cell lines are expressed as a percentage of this highest activity); gray box, not measured. Red, breast cancer lines; green, melanoma cancer lines; black, NCI/ADR is of unknown origin.

FIGS. 4A-4C. Correlation between the activity of the membrane-associated hydrolase KIAA1363 and the invasiveness of human cancer cell lines. (A-C) Levels of active KIAA1363 present in cancer cell membrane proteomes as measured by ABPP (Left), and cancer cell invasiveness as measured by matrigel invasion assays (Right). Results expressed as number of invading cells refers to average number of invading cells per 8 fields counted (n=3-4 for each cell line). (A) Breast carcinoma lines. (B) Melanoma lines. (C) Ovarian carcinoma lines.

FIGS. 5A-5W. FIGS. 5A-5W show bar graphs corresponding to a serine hyrdolase activity identified in the panel of cancer cell lines. For the secreted and membrane enzyme activities, representative P values are shown (calculated by Tukey's honestly significant difference test, where a P value of <0.05 is considered significant).

### DETAILED DESCRIPTION OF THE INVENTION

Methods and compositions are provided concerning enzyme profiles of cells, particularly tumor cells, where the sample being analyzed will usually be from a single source. It is shown that by analyzing for active enzymes in a cell sample, useful information can be derived that can be applied in a number of ways. Cells can be analyzed as to whether they are neoplastic and, if neoplastic, the tumor cells can be evaluated as to their origin, invasiveness or aggressiveness, hormone status for steroid responsive tumors, as well as response to therapy. The cellular contents, which may be fractionated and deglycosylated, are reacted with activity-based probes that preferentially react with the active site of enzymes. The probes have ligands that allow for manipulation of the resulting conjugate for determination and quantitation of the enzyme of the complex.

The subject method provides a new way of analyzing cells in relation to their neoplastic condition. The method employs single or groups of probes that are specific for specific members of a class of enzymes, where the enzymes are found to be up- or down-regulated in their active form based on the nature and the environment of the cells. By analyzing the cells as to a particular cluster of enzymes, usually at least about 3, more usually at least about 5, and not more than about 30, more usually not more than about 20, preferably not more than about 10, patterns can be observed in relation to the nature of the cell and its neoplastic condition. The amount of the individual conjugates can be determined, so that comparisons can be made of the amount of each target enzyme present. Of particular interest are members of the hydrolase family, more particularly, the serine hydrolase family.

The method involves preparing the cells for analysis. This will depend upon whether the cells are primary cells, cells grown in culture, cell lines, or other cellular composition. To expand the number of available cells, the cells may be grown in an appropriate growth medium, primarily conventional growth media such as RPMI-1640 with 10% fetal calf serum under conventional temperature and environmental conditions, followed by growth in serum-free media, generally over a period of 1 to 4 days. Conveniently, the cells are initially grown to from about 75 to 85% confluence before growth in the serum-free medium. The conditioned medium resulting from the second phase can be used for analysis of secreted proteins. After centrifugation or other means for removal of debris, the protein from the debris-freed medium, e.g. supernatant, is precipitated and suspended in an appropriate buffer medium. Various buffer media may be used that do not interfere with the reaction between the probe and the serine hydrolases, such as Tris-HCl (50 mM, pH 7.5), and the like. After desalting, cell pellets are dispersed and homogenized in a conventional buffer, followed by separation of the medium into the soluble cellular fraction and the membrane pellet. The membrane pellet is then solubilized. In this manner, one may obtain three fractions: secreted protein; soluble protein; and membrane bound protein, from the cells. In many instances only one or more of these fractions will be employed to obtain the desired information about the cells. Initially, one may wish to analyze all three fractions to provide a standard that may then be used for comparison with other cell samples.

When using primary cells, frequently they will come from a biopsy for a solid tumor and will usually be a mixture of both normal and abnormal cells. If one wishes to assay for both up- and down-regulation, then it will be necessary to separate the cells. Various techniques may be used to separate the different types of cells by employing surface markers specific for the type of cell. Panning, fluorescence activated cell sorting, or other separation technique may be employed to enrich for one type of cell. One can assay the normal cells for their enzyme profile to obtain an indication of what level of the different enzymes is present. By identifying an enzyme that is present in the normal cells and absent in the abnormal cells, one can use this enzyme as a standard for the proportion of normal cells present in a mixture. This will allow for some measure of the amount of down regulation in the abnormal cells.

In some instances intact cells can be employed for the determination, where the probe(s) that are employed are introduced intracellularly. This can be the result of using probes that can cross the cell membrane, employing an agent that permeabilizes the cells without changing the status of the cells during the time of the measurement, lipofection, or other convenient means.

One or more of the cell fractions (including intact cells as a fraction) is independently combined with the probe(s). For quantitation, a standard is used, conveniently standardizing the amount of protein in the fraction. The reaction is performed under standardized conditions to allow for comparison between samples from the same or different cells. The amount of protein in the reaction mixture will generally be in the range of about 0.01 to 5 mg/ml, usually 0.5 to 2 mg/ml. Various buffers may be used to obtain the desired protein concentration, such as those described above. The temperature for the reaction will generally be in the range of about 20 to 40° C., where the time for the reaction will depend on whether intact cells or cell fractions are employed, the time generally being in the range of about 5 to 120 min, usually about 15 to 90 min, desirably substantially to completion. The probe(s) usually will be used in stoichiometric excess, generally at least about 1.5 fold excess and may be 2-fold excess or more, usually less than about 10-fold excess. The excess will be related to the time of the reaction, as the probes have reactive functionalities that at high concentrations and extended periods of time, non-specific reactions will increase, so as to interfere with the analysis. By running a few standard samples, one can optimize the conditions to minimize the background while providing a robust result.

After the reaction is completed, the conjugates of the probes and protein targets will be analyzed. As will be discussed in greater detail below, the probes have a ligand that allows for manipulation of the conjugates, either for sequestering the conjugates or detecting the conjugates or both. The probes may be analyzed by electrophoresis, using gel electrophoresis, capillary electrophoresis or microfluidic electrophoresis, mass spectrometry, e.g. MALDI-TOF, microcapillary liquid chromatography-electrospray tandem MS, or other technique.

To enhance the analysis, the conjugates may be deglycosylated using an appropriate glycosidase, such as PGNaseF, under conventional deglycosylation conditions indicated by the supplier.

The results obtained from analyzing the conjugates may then be organized in a manner that allows for ready comparisons and differentiation between samples. One technique that finds utility is cluster analysis. One applies a hierarchical clustering algorithm to the samples using the Pearson correlation coefficient as the measure of similarity and average linking clustering (Cluster program: Ross, et al., Nat. Genet. 24, 227-35, 2000; Eisen, et al., Proc. Natl. Acad. Sci. USA 95, 14863-68, 1998). For each enzyme activity, averaged cell sample values are compared to identify the cell sample that expressed the highest level of a particular enzyme activity. The activity levels may then be expressed as a percentage of this highest activity to normalize the data sets. As data sets are built up from cell samples, both primary and cell lines, the cluster analysis can be modified in light of new data that provides a new maximum for a particular enzyme, so that one may have cluster analysis within a given group of samples as well as cluster analysis extending over many samples and groups of samples. Cluster analysis can also be applied as to the individual fractions and pair-wise combinations, so as to extract the greatest amount of information from the cell samples in relating the samples to each other and standards. As the data increases from additional numbers of samples, the Clustergrams can be used to rapidly identify the similarities between samples, origin of the cells, aggressiveness and invasiveness, preferential therapies and how the tumor has responded to a course of treatment.

An important aspect of this invention is that the probes react with active enzymes. By an “active enzyme” is intended an enzyme, in its normal wild-type conformation, e.g. a catalytically active state, as opposed to an inactive state. The active state allows the enzyme, to function normally. An inactive state may be as a result of denaturation, inhibitor binding, either covalently or non-covalently, mutation, secondary processing, e.g. phosphorylation or dephosphorylation, absence of binding to another protein, etc. Functional states of enzymes as described herein may be distinct from the level of abundance of the same enzymes. An active site is an available wild-type conformation at a site that has biological activity, such as the catalytic site of an enzyme or a cofactor-binding site, or other site where binding of another entity is required to provide catalytic activity. In many instances, one is interested in knowing the level of availability of such sites.

Activity-based probes (ABPS) are provided for specific reaction with the active site of one or more target enzymes, where the target protein is a member of a class of proteins, particularly enzymes, for detection of the presence and quantitation of one or more active members. A single fABP (fluorescent labeled ABP) or mixture of fABPs may be used, where the electrophiles may be different, the environments may be different and the fluorescent labels may be different, so as to provide different profiles. The probes may be divided into four characteristics, where the same component may serve two functions and two or more components may together serve a single or multiple functions: (1) a functional group (F) that specifically and covalently bonds to the active site of a protein; (2) a fluorescent label (F1) 3) a linker L, between the F1 and the F; and 4) binding moiety or affinity moiety or label, that may be associated with or part of the linker region and/or the functional group (R) and with serine hydrolases, the binding affinity of the functional group is influenced by the nature of the linker. F and L may be combined to provide an affinity label, as well as the reactive functionality and the linker.

A linker is a bond or chemical group used to link one moiety to another, serving as a divalent bridge, where it provides a group between two other chemical moieties. “Binding or affinity moiety” refers to a chemical group, which may be a single atom, that is conjugated to the reactive functional group or associated with the linker, as a side chain or in the chain of the linker, and provides enhanced binding affinity for protein targets and/or changes the binding profile of the probe. To the extent that the probe enjoys specificity for active sites of target enzymes, various portions of the probe molecule may contribute to the binding profile of the probe molecule. Fluorescer refers to a fluorophore that can be excited when in a gel and the emitted light desirably used to quantitate the amount of fluorophore, in effect, the amount of protein, present in the excitation light pathway.

The fABP has an affinity for an enzyme active site, which, while it may be specific for the active site of a particular enzyme, will usually be shared by a plurality of related enzymes

Exemplary Fs as used in an fABP of the invention include an alkylating agent, acylating agent, ketone, aldehyde, sulphonate, photoaffinity or a phosphorylating agent. Examples of particular Fs include, but are not limited to fluorophosphonyl, fluorophosphoryl, fluorosulfonyl, alpha-haloketones or aldehydes or their ketals or acetals, respectively, alpha-haloacyls and nitriles, sulfonated alkyl or aryl thiols, iodoacetylamide group, maleimides, sulfonyl halides and esters, isocyanates, isothiocyanantes, tetrafluorophenyl esters, N-hydroxysuccinimidyl esters, acid halides, acid anhydrides, iminoethers, unsaturated carbonyls or cyano, alkynes, hydroxamates, hemiacetals, alpha-halomethylhydroxamates, aziridines, epoxides, partiocularly spiroexpoxides, azides, or arsenates and their oxides. Sulfonyl groups may include sulfonates, sulfates, sulfinates, sulfamates, etc., in effect, any reactive functionality having a sulfur group bonded to two oxygen atoms. Epoxides may include aliphatic, aralkyl, cycloaliphatic and spiroepoxides, the latter exemplified by fumagillin, which is specific for metalloproteases.

Specificity can be achieved by having groups as part of the active functionality, e.g. sulfonate or sulfate esters, fluorophosphonates, substituted spiroepoxides, etc., where the substituents may be aliphatic, alicyclic, aromatic or heterocyclic or combinations thereof, aliphatically saturated or unsaturated, usually having fewer than 3 sites of unsaturation. Illustrative groups include alkyl, heterocyclic, such as pyridyl, substituted pyridyl, imidazole, pyrrole, thiophene, furan, azole, oxazole, aziridine, etc., aryl, substituted aryl, amino acid or peptidyl, oligonucleotide or carbohydrate group. Many of the functionalities are found in the literature, such as fluorophosphonates, spiroepoxides, sulfonates, olefins, carbonyls, and the like. See, for example, Cravatt B F & Sorensen E J “Chemical strategies for the global analysis of protein function”, Curr Opin Chem Biol December 2000; 4(6):663-8. Of particular interest for profiling neoplastic cells are the fluorophosphonates.

fABPs of the subject invention may be illustrated by the following formula:

FPO₂—L—FL

where the symbols are:

FPO₂ intends fluorophosphonyl;

L is a linker of from 2 to 20, usually 2 to 16, carbon atoms and may be aliphatic, aromatic, alicyclic, heterocyclic or combination thereof, particularly aralkyl, and may include from about 0 to 6 heteroatoms in the chain, e.g. O, S, N and P, such as phenylalkylene, phenylpoly(oxyalkylene), alkylene, poly(oxyalkylene), where the oxyalkylene will usually be of from 2 to 3 carbon atoms; and

F1 is a fluorescent moiety.

The linker group, while potentially it can be a bond, is preferred to be other than a bond. Since in many cases, the synthetic strategy will be able to include a functionalized site for linking, the functionality can be taken advantage of in choosing the linking group. The choice of linker, as with the choice of an R group, has been shown to alter the specificity of an fABP. For example, an alkylene linker and a linker comprising polyethylene glycols (“PEG”), have distinct specificities and provide distinct protein profiles. Thus, one of skill in the art can select the linker portion of the fABP in order to provide additional specificity of the fABP for a particular enzyme or enzyme class.

Linker groups include among others, ethers, polyethers, diamines, ether diamines, polyether diamines, amides, polyamides, polythioethers, disulfides, silyl ethers, alkyl or alkenyl chains (straight chain or branched and portions of which may be cyclic) aryl, diaryl or alkyl-aryl groups, having from 0 to 3 sites of aliphatic unsaturation. While normally amino acids and oligopeptides are not preferred, when used they will normally employ amino acids of from 2-3 carbon atoms, i.e. glycine and alanine. Aryl groups in linkers can contain one or more heteroatoms (e.g., N, O or S atoms). The linkers, when other than a bond, will have from about 1 to 60 atoms, usually 1 to 30 atoms, where the atoms include C, N, O, S, P, etc., particularly C, N and O, and will generally have from about 1 to 12 carbon atoms and from about 0 to 8, usually 0 to 6 heteroatoms. The atoms are exclusive of hydrogen in referring to the number of atoms in a group, unless indicated otherwise.

Linkers may be varied widely depending on their function, including alkyleneoxy and polyalkyleneoxy groups, where alkylene is of from 2-3 carbon atoms, methylene and polymethylene, polyamide, polyester, and the like, where individual monomers will generally be of from 1 to 6, more usually 1 to 4 carbon atoms. The oligomers will generally have from about 1 to 10, more usually 1 to 8 monomeric units. The monomeric units may be amino acids, both naturally occurring and synthetic, oligonucleotides, both naturally occurring and synthetic, condensation polymer monomeric units and combinations thereof. Alteration in the linker region has been shown to alter the specificity of the fABP for a class of enzymes.

The fluorescers may be varied widely depending upon the protocol to be used, their effect on the specificity of the probe, if any, the number of different probes employed in the same assay, whether a single or plurality of lanes are used in the electrophoresis, the availability of excitation and detection devices, and the like. For the most part, the fluorescers that are employed will absorb in the ultraviolet and visible range and emit in the visible and infra red range, particularly emission in the visible range. Absorption will generally be in the range of about 350 to 750 nm and emission will generally be in the range of about 400 to 900 nm. Illustrative fluorophores include xanthene dyes, naphthylamine dyes, coumarins, cyanine dyes and metal chelate dyes, such as fluorescein, rhodamine, rosamine, BODIPY, dansyl, lanthanide cryptates, erbium. terbium and ruthenium chelates, e.g. squarates, and the like. The literature amply describes methods for linking the fluorescers through a wide variety of functional groups to other groups. The fluorescers have functional groups that can be used as sites for linking. The fluorescers that find use will normally be under 2 kDal, usually under 1 kDal.

Of interest is the use of matched dyes as described in U.S. Pat. No. 6,127,134, which is concerned with labeling proteins with dyes that have different emissions, but have the same migratory aptitude in electrophoresis. By same migratory aptitude is intended that the dyes when bound to the same molecule, e.g. protein, at the same site in the same way, the resulting conjugates will form a substantially superimposable band when the compounds are subjected to gel electrophoresis. Of particular interest are the cyanine dyes disclosed therein, being selected in '134 because of their positive charge, which matches the lysine to which the cyanine dyes bind. In addition there is the opportunity to vary the polyene linker, while keeping the molecular weight about the same with the introduction of an alkyl group in the shorter polyene chain dye to offset the longer polyene. Also described are the BODIPY dyes, which lack a charge. The advantage of having present a combination of two dyes that similarly affect the migration of the enzyme is particularly applicable when comparing the native and inactived samples, although this would require that in the inactivated sample at least a portion of the enzyme is monosubstituted.

In some instances, it may be desirable to have a ligand bound to the fABP to allow all of the fABPs, conjugated or unconjugated to be captured and washed free of other components of the reaction mixture. This can be of particular interest where the protein bound to the fABP is partially degraded, leaving an oligopeptide that is specific for the protein and can be analyzed with a mass spectrometer. For mass spectrometry or for other purposes, the ABPs, where the fluorescer may be present or absent, may be labeled with low abundance isotopes, radioactive or non-radioactive. Also, the ligand allows for a cleaner sample to be used for electrophoretic separation, by capture, wash and release. The ligand will generally be under about 1 kDal and biotin is a conventional ligand, particularly analogs such as dethiobiotin and iminobiotin, which can be readily displaced from strept/avidin by biotin. However, any small molecule will suffice that can be captured and released under convenient conditions. The ligand will be placed distant from the functional group, generally by a chain of at least about 3 atoms, usually at least about 4 atoms.

The enzymes found by the fluorophosphonate probes with neoplastic cells include the serine hydrolases: complement component 1s, PAF acetylhydrolase, particularly isoform 1b, fatty acid amide hydrolase (“FAAH”), palmitoyl-protein thioesterase (“PPT-2”), butyryl-cholinesterase (“BCHE”), p25 kDa hydrolase, cathepsin A, phosphatidylserine-specific phospholipase 1 (“PS-PL1”), urokinase type plasminogen activator (“uPA”), esterase D, membrane hydrolase, lower glycosylated form (“KIAA1363 Lower”) and upper glycosylated form (“KIAA1363 Upper”), platelet-activating factor acetylhydrolase 2 (“PAF-AH 2”), p26 kDa cytosolic hydrolase, fatty acid synthase, acyl-peptide hydrolase (“APH”), dipeptidyl peptidase VIII (“DPP8”), lysophospholipase 1 (“Lyso-PL1”), soluble alpha/beta hydrolase (FLJ11342), soluble alpha/beta hydrolase (PP1226), peroxisomal long-chain acyl CoA thioesterase (“PTE”) and angiotensinase C. (Those enzymes indicated by letters and a number of at least 3 digits are previously unisolated enzymes designated by est's.)

The KIAA1363 enzyme is characterized by being a membrane protein having at least two different glycosylated forms with different specificities for neoplastic cells, being upregulated in neoplastic cells. It is found in both breast and melanoma cancer cell lines and is particularly abundant in MUM-2B. As expected, the protein is membrane associated, being glycosylated, is an invasive marker when highly glycosylated. It reacts with fluorophosphonate specifically in the active form, regardless of the level of glycosylation. The protein appears to be limited to embryonic cells, cancer cells and the nervous system in its expression profile. As such it is a desirable marker in that it is absent in most cells in the body and that drugs that cannot cross the blood-brain barrier will not interfere with its function in the nervous system.

A partial sequence includes:

| | |
|---|---|
| mrsscvlltalvalatyyvyiplpgsvsdpwklmlldatfrgaqqvsnlihylglshhllalnfiivsfgkksawssaqv | (SEQ ID: NO. 1) |
| kvtdtdfdgvevrvfegppkpeeplkrsvvyihgggwalasakiryydelctamaeelnavivsieyrlvpkvyfpeqih | |
| dvvratkyflkpevlqkymvdpgricisgdsaggnlaaalgqqftqdaslknklklqaliypvlqaldfntpsyqqnvnt | |
| pilpryvmvkywvdyfkgnydfvqamivnnhtsldveeaaavrarlnwtsllpasftknykpvvqttgnarivqelpqll | |
| darsapliadqavlqllpktyiltcehdvlrddgimyakrlesagvevtldhfedgfhgcmiftswptnfsvgirtrnsy | |
| ikwldqnl. | |

Peptides identified by MALDI-MS from FP-isolated protein (approximately 25% total coverage) MH+peak

| | | |
|---|---|---|
| 1079.59 | (K)LMLLDATFR(G) | (SEQ ID NO: 2) |
| 1394.80 | (R)IVQELPQLLDAR(S) | (SEQ ID NO: 3) |
| 1447.77 | (K)NYKPVVQTTGNAR(I) | (SEQ ID NO: 4) |
| 1501.78 | (K)VYFPEQIHDVVR(A) | (SEQ ID NO: 5) |
| 1622.89 | (R)VFEGPPKPEEPLKR(S) | (SEQ ID NO: 6) |
| 3426.84 | (K)LQALIYPVLQALDFNTPSYQQNVNTPILPR(Y). | (SEQ ID NO: 7) |

KIAA1363 has not been reported as an isolated protein and cannot be found in the data banks describing known human proteins. As indicated above, it can be used as a target for the treatment of neoplastic cells. It can also be used for the preparation of antibodies, both antisera and monoclonal antibodies, as described below. It may also be used to prepare labeled derivatives, both fragments and the intact protein, glycosylated and deglycosylated. Various labels may be used, such as fluorescers, radioactive labels, enzyme fragments, particles, molecular dots, etc. The methods for conjugating labels to KIAA1363 are well known in the literature and need not be described here.

These proteins are readily purified to at least about 50% purity (based on total protein), usually at least about 75% purity, and desirably at least about 90% purity to totally pure, using one or more conventional methods for protein purification, such as SDS-PAGE, liquid chromatography, particularly HPLC, or capillary electrophoresis.

These proteins serve as targets for candidate compounds to be used for determining the activity of candidate compounds for inhibiting the enzyme activity. Various techniques can be used for evaluating candidate compounds. In one method, one may use the probes as competitors for the candidate compound for binding to the active site of the enzyme. By combining the enzyme, the probe and the candidate compound in an appropriately buffered medium, one determines the change in conjugate formation in the presence and absence of the candidate compound. Alternatively, one may combine the candidate compound and enzyme substrate with the enzyme and determine the change in turnover in the presence and absence of the candidate compound. Other techniques may also be used, as appropriate.

The subject KIAA enzymes can be used for production of antisera and monoclonal antibodies in accordance with conventional procedures. Mammalian hosts may be immunized with the enzyme, usually in the presence of an adjuvant, employing conventional regimens of injections, waiting 2-4 weeks, bleeding to determine titer, followed by further immunizations to obtain high titer antisera. For monoclonal antibodies, the proteins can be used to immunize mice or other convenient mammalian host, splenocytes isolated and immortalized and the resulting hybridomas screened for affinity for the proteins. These techniques are well known and described in texts. See, for example, Antibodies: A laboratory manual, eds. David Lane and Ed Harlow, Cold Spring Harbor Laboratories, Cold Spring Harbor, N.Y. Novel enzyme associations have been found with the use of the probes and tumor cell lines, identifying levels of active hydrolase enzymes in the proteins, where as indicated above, the enzymes can be used to differentiate origin, aggressiveness, steroid response and invasiveness. The secreted enzymes have a restricted distribution. BCHE and cathepsinA are secreted by melanoma cells but are not detected with breast carcinomas, with the exception of the estrogen receptor negative [ER(−)] cells that were studied, which secreted high levels of these enzymes. It may be that the latter cells are misinterpreted as breast cancer and may be melanoma cells based on a microarray analysis (Ross, et al., Nat. Genet. 24, 227-35, 2000). These enzymes are down regulated with a highly invasive melanoma line. Urokinase appears to be a marker for aggressiveness, being secreted at upregulated levels in MUM-2B, MDA-MB-231 and NCI/ADR.A subset of the secreted enzymes have been represented on cDNA microarrays previously (Ross, et al., 2000, supra; and Bittner, et al., 2000, supra). Urokinase is determined at very different levels between the two analytical methods. It appears that the transcriptional level is substantially different from the activity level, possibly being explained by the presence of PAI-1, a urokinase inhibitor. This observation points to the significance of being able to identify enzyme activity rather than transcription levels or total enzyme protein.

The membrane-associated serine hydrolase activities also have restricted patterns of distribution among cancer cells. FAAH is detected exclusively in breast cancer cells, where the level varies with different cancer cells. PPT2 is upregulated in most melanoma as compared to breast carcinomas. KIAA1363 is upregulated in invasive cancer cells, with the upper glycosylated form being associated with invasiveness among breast carcinomas.Among the soluble proteins PAF acetylhydrolase Ib, beta subunit is found primarily in ER(+) breast carcinomas.

The cancer cells studied when looked at by cluster analysis of the active serine hydrolases identified, fall into three main categories: a melanoma cluster, a breast carcinoma cluster, and an invasive cluster. By determining the level of at least one enzyme, usually of at least two of the enzymes, conveniently at least four of the enzymes, generally from about 2 to 10, more generally from about 2 to 6, enzymes, one can determine the origin of the tumor cells, hormone status, invasiveness or metastatic potential and response to treatment. Of particular interest among the family of enzymes determined to be present are the markers associated with invasiveness: urokinase, KIAA1363, BChE, and cathepsin A. Other markers that can be used for the other purposes are PAF acetylhydrolase Ib, beta subunit, PPT2, FAAH, p25, p26, angiotensinase C, and esterase D to mention only a few set forth above.

The subject methodology may be applied in conjunction with other techniques to obtain profiles, such as microarrays for determining transcription levels or total protein levels. By comparing the results from the different methodologies, one can ascertain the level of transcription, the total amount of protein and the fraction that is active. In this way, the biopsies may be analyzed to determine the origin of the tumor, the status of the tumor, likely response to a therapeutic regimen and the actual response. The following examples are intended to illustrate but not to limit the invention in any manner, shape, or form, either explicitly or implicitly. While they are typical of those that might be used, other procedures, methodologies, or techniques known to those skilled in the art may alternatively be used.

### EXAMPLE 1

Isolation of FP-labeled proteins was achieved using biotinylated FPs and an avidin-based affinity purification procedure as described previously (Kidd, et al., Biochemistry 40, 4005-15, 2000). Avidin-enriched FP-labeled proteins were separated by SDS-PAGE and the protein bands excised, digested with trypsin, and the resulting peptides analyzed by a combination of matrix assisted laser desorption mass spectrometry (MS)[Voyager-Elite time-of-flight MS instrument (PerSeptive Biosystems)] and microcapillary liquid chromatography-electrospray tandem MS (Agilent 1100 HPLC combined with a Finnigan LCQ MS). The MS data were used to search public databases to identify the FP-labeled proteins as described previously (Kidd, et al., 2000, supra).The MS data for one FP-labeled proteins did not match any proteins in the databases, and therefore, this protein has been designated as previously “unidentified hydrolase.”

The protein band gel pieces were cut into several small pieces and destained with MeOH, washed with 100 mM ammonium bicarbonate in 30% acetonitrile a few times, then digested with trypsin (100 ng) in 3 mM Tris-HCl) at pH 8, 37° C. for over night. The tryptic peptides were extracted out of the gel using 50% acetonitrile/0.1% TFA, concentrated to 10 μl, and subjected to Nano-LCMS to be analysis.

Nano-Capillary HPLC-Tandem Mass Spectrometry (MS/MS):

The nano-LCMS/MS experiment was performed on a combination system of Agilent 1100 capillary HPLC/Micro Auto-sampler (Agilent Technologies, Palo Alto, Calif.) and Finnigan LCQ DecaXP ion trap mass spectrometer (Finnigan, San Jose, Calif.).

LC Separation:

A 3 μl of digested sample was mixed with 3 μl of 5% Acetic Acid and loaded on a 100 μm fused silica capillary C₁₈ column. A sixty-minutes gradient of 5-95% solvent B (A: H₂O/0.1% Formic Acid, B: MeCN/0.08% Formic Acid) and a 500 nl/min column flow rate was used to separate the tryptic peptides in the digested sample. Peptides eluted out from the column were directly injected into LCQ DecaXP mass spectrometer to be analyzed.

Mass spectrometry:

The heated desolvation capillary in the mass spectrometer was held at 200° C., the spray voltage was set at 2.0 kV and the capillary voltage was set at 30 V. During the experiment, the mass spectrometer was set to alternate between MS and MS/MS mode. The scan range for MS was set at m/z 400-1600. The MS/MS spectra were acquired in dependent scan mode with an initiating minimum MS signal at 2×10⁵ counts, and a 35% normalized collision energy. The scan range for MS/MS is varied from 80-2000 depending on the precursor ion.

Protein Identification:

The ion masses and the fragmentation information generated by nano-LCMS/MS experiment were analyzed and converted into peptide masses and sequence information with TurboSEQUEST, which is protein identification software. With this program, searching against the protein database with that information from the spectra then identified proteins.

FAAH Enzyme Activity Assays.

FAAH enzyme activity assays were conducted using ¹⁴C-oleamide as a substrate as described previously (Cravatt, et al., Nature 384, 83-7, 1996).

Cluster Analysis of Proteomic Profiles.

For each serine hydrolase activity, averaged cell line values were compared to identify the line that expressed the highest level of this enzyme activity, which was defined as 100%. The activity levels of the rest of the cell lines were expressed as a percentage of this highest activity to normalize the data sets. We then applied a hierarchical clustering algorithm to the cell lines using the Pearson correlation coefficient as the measure of similarity and average linkage clustering (Cluster program, Ross,et al., Nat. Genet. 24, 227-35, 2000; Eisen, et al., Proc. Natl. Acad. Sci. USA 95, 14863-68, 1998). Additional cluster analyses were performed on enzyme activity profiles of the secreted, membrane, and soluble proteomes separately and in all of their respective pair-wise combinations. Of these six additional cluster analyses, only the “membrane+secreted” analysis produced a dendrogram with increased distances among the three major clusters observed in the total serine hydrolase activity analysis.

TABLE 1
Relative activities of secreted and membrane serine
hydrolases in invasive/non-invasive cancer cell
line pairs

| Serine Hydrolase | MDA-MB-231/MCF7 | MUM-2B/MUM-2C |
|---|---|---|
| Invasive Markers | | |
| uPA | >50 | >50 |
| K1AA1363 upper | 8.3 | 11.1 |
| Esterase D | 3.7 | 6.7 |
| Angiotensinase | 1.5 | 12.6 |
| Non-Invasive Markers | | |
| BCHE | — | 0.1 |
| Cathepsin A | — | 0.04 |
| PPT-2 | — | 0.1 |
| 25 kDa Hydrolase | — | 0.07 |
| PAF-AH2 | 0.3 | 0. |
| FAAH | <0.02 | — |

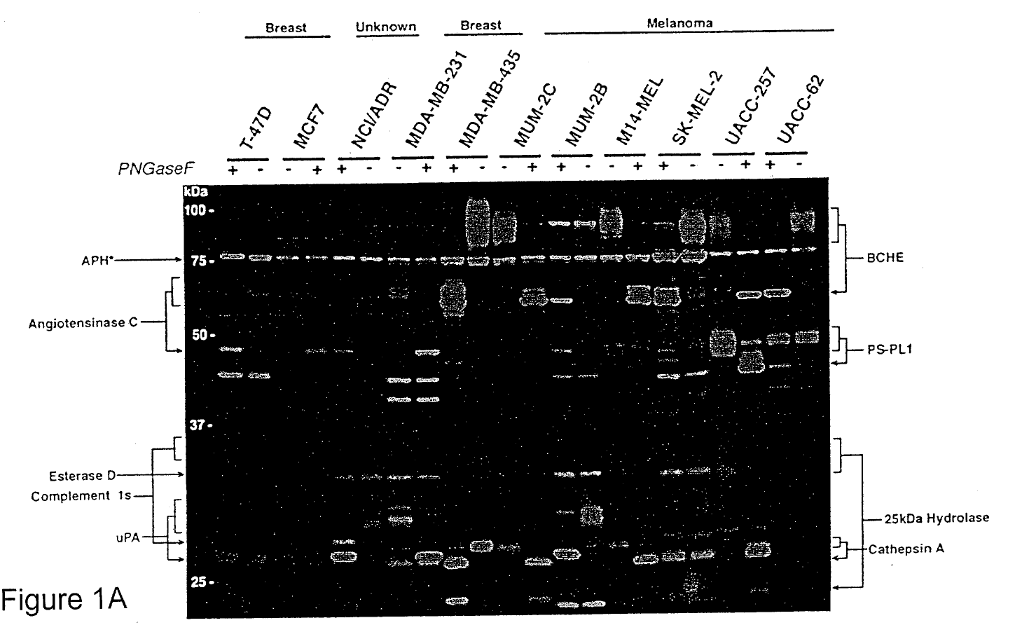

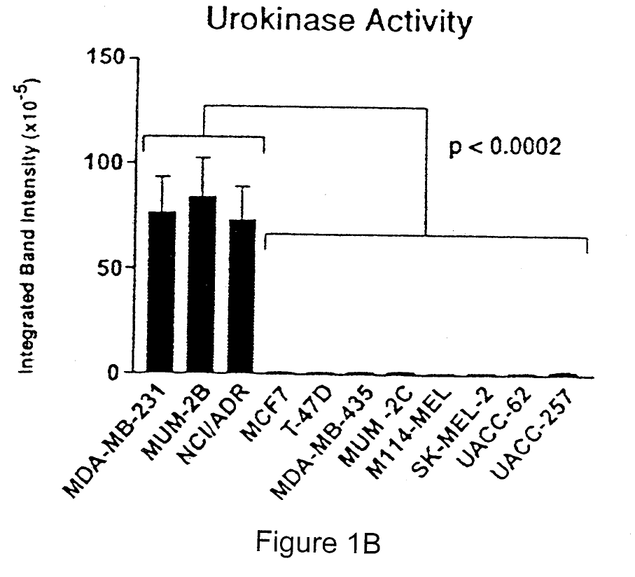

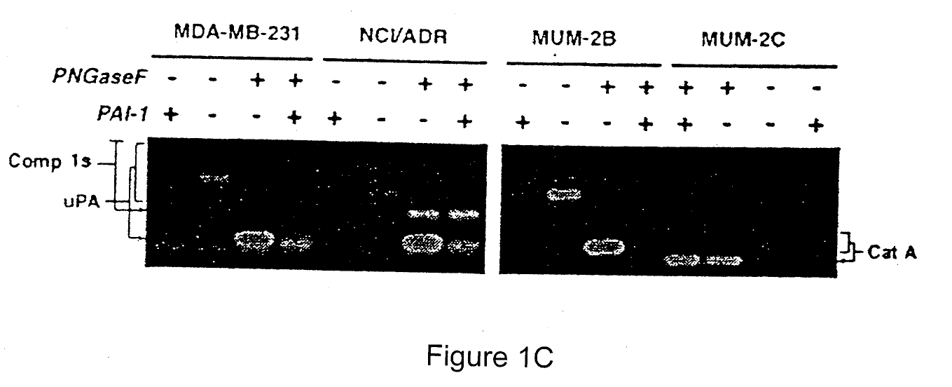

In the subject invention a panel of human cancer cell lines for comparative analysis by ABPP were employed based on the following criteria: 1) they represent multiple lines derived from at least two distinct types of cancer, and therefore permit the comparison of proteomic expression patterns both within and between cancer classes, 2) they exhibit a diverse range of well characterized cellular properties, including differences in hormone status, invasiveness, and metastatic potential, and 3) they have previously been analyzed with gene expression microarrays, and therefore allow for a direct comparison between proteomic data and trancriptional profiles (Scherf, et al., Nat. Genet 24, 236-44, 2000; Bittner, et al., Nature 406, 536-40, 2000). Proteomes from each cell line were separated into three cellular fractions (secreted, membrane, and soluble) prior to treatment with a rhodamine-tagged FP probe. Fluorescently labeled proteins were then separated by SDS-PAGE and visualized in-gel using a flatbed laser-induced fluorescence scanner. Membrane/soluble and secreted proteomes were tested in duplicate from two and three independent cell culture preparations, respectively, resulting in 4-6 distinct serine hydrolase activity profiles for each proteomic specimen. The integrated band intensities for each enzyme activity were averaged to provide the results shown in FIGS. 1-3 and Table 1. FIG. 1A shows a representative in-gel fluorescence analysis of the secreted serine hydrolase activity profiles of the cancer cell lines. Several enzyme activities appeared as faint, diffuse bands, suggesting that they existed in a highly glycosylated state. Therefore, a portion of each FP-labeled proteome was deglycosylated prior to separation by SDS-PAGE, resulting in a striking increase in the resolution of these proteins (see, for example, the serine proteases complement 1s and angiotensinase C).

Most of the hydrolase enzymes exhibited a restricted distribution among the cell lines. For example, two enzyme activities, butyrylcholinesterase (BCHE) and cathepsin A, were secreted by most melanoma lines, but were not detected in any of the breast carcinomas with the exception of the estrogen receptor negative [ER(−)] line MDA-MB-435, which secreted high levels of these enzymes. Interestingly, as previously indicated the MDA-MB-435 line was recently suggested to be a misclassified cancer line based on cDNA microarray analyses that revealed its transcriptional profiles clustered more closely with those of melanomas than breast carcinomas. Thus, BCHE and cathepsin A appeared to represent secreted enzyme activities characteristic of melanomas. Both of these proteins were dramatically down regulated in the highly invasive melanoma line,MUM-2B. Instead, MUM-2B cells secreted high levels of active urokinase, a serine protease that was also upregulated in the other aggressive lines examined, including the ER(−) breast carcinoma MDA-MB-231 and the multi-drug resistant NCI/ADR line (unknown origin).

A subset of the secreted serine hydrolases were represented on cDNA microarrays previously used to analyze the gene expression patterns of this panel of cancer cell line (Ross, et al., 2000, supra; and Bittner, et al., 2000, supra). Therefore, for these enzymes, a direct comparison could be made between their activity and transcriptional profiles. Some of the serine hydrolases, like BCHE and cathepsin A, exhibited activity profiles that closely resembled the expression pattern of their transcripts. In contrast, other enzymes, like urokinase, displayed activity profiles that markedly differed from their transcriptional profiles. Whereas approximately equal levels of active urokinase were observed in the MDA-MB-231 and NCI/ADR lines, four to eight-fold more urokinase transcript was observed in the former cell line 5. Interestingly, transcript levels of the endogenous urokinase inhibitor PAI-1 were also higher in the MDA-MB-231 line relative to the NCI/ADR line, indicating that this inhibitor may act to buffer the urokinase activity of MDA-MB-231 cells, reducing it a level that matches the amount of active protease secreted by NCI/ADR cells. Consistent with this notion, the addition of excess PAI-1 to these cancer proteomes blocked over 85% of the observed urokinase activity, without affecting the activity of other proteases. (FIG. 1C)

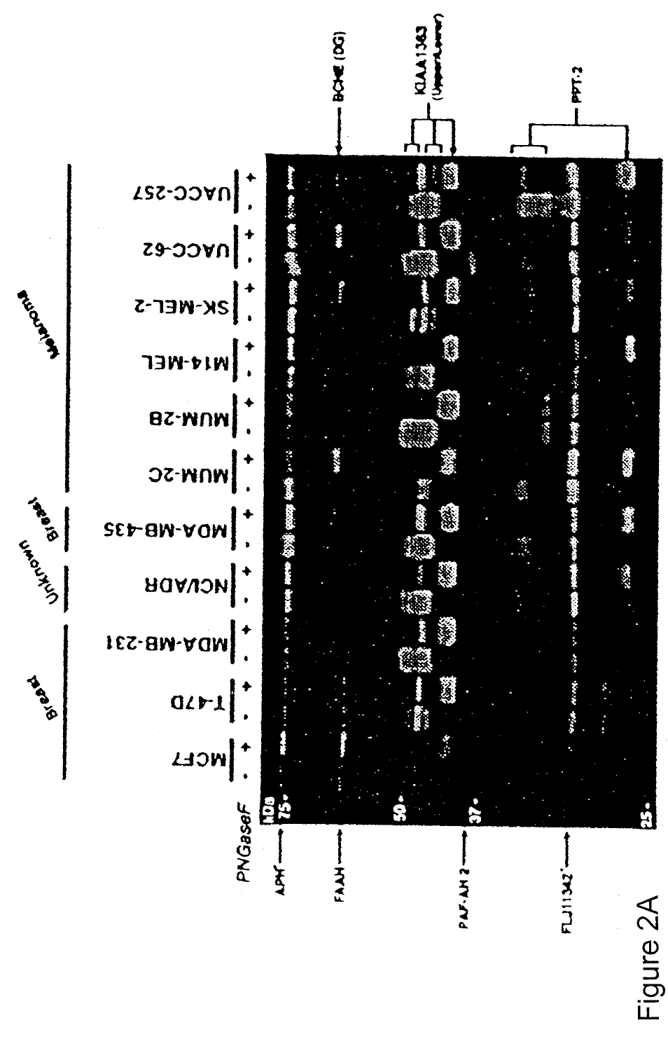

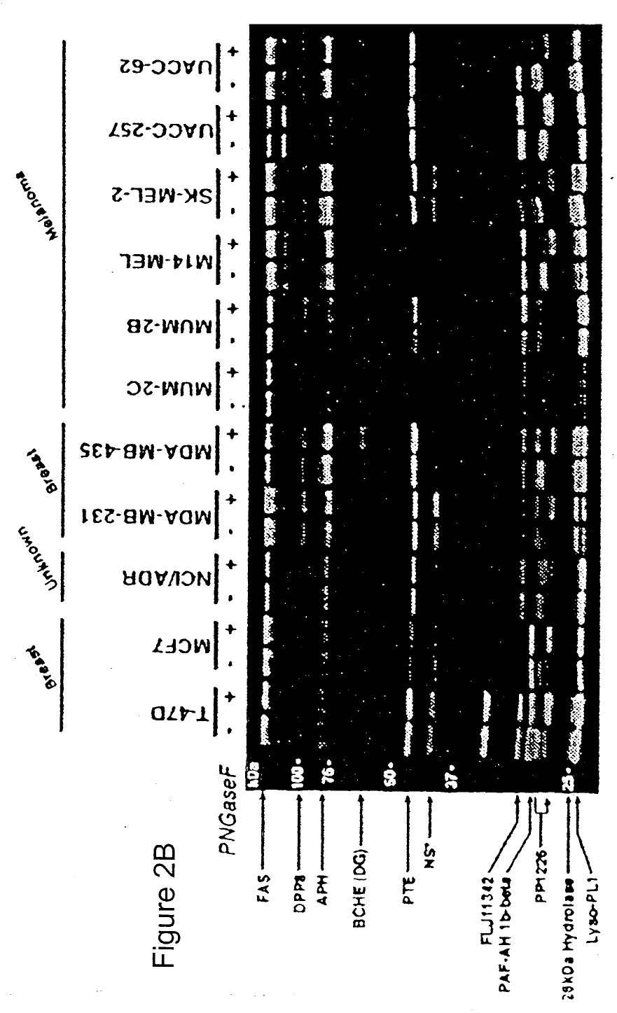

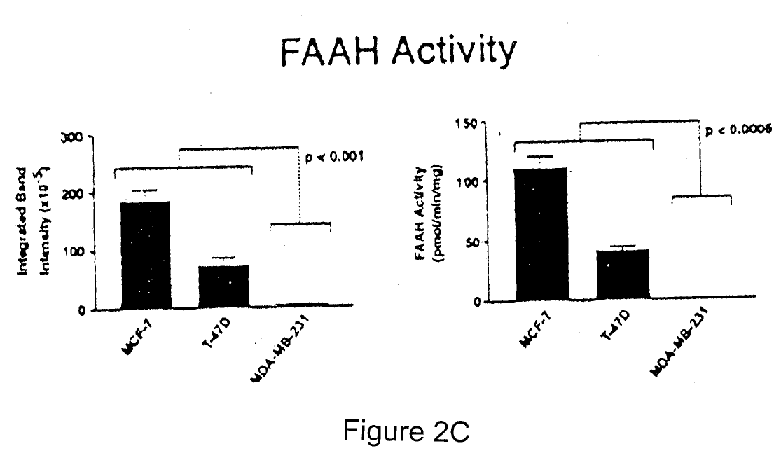

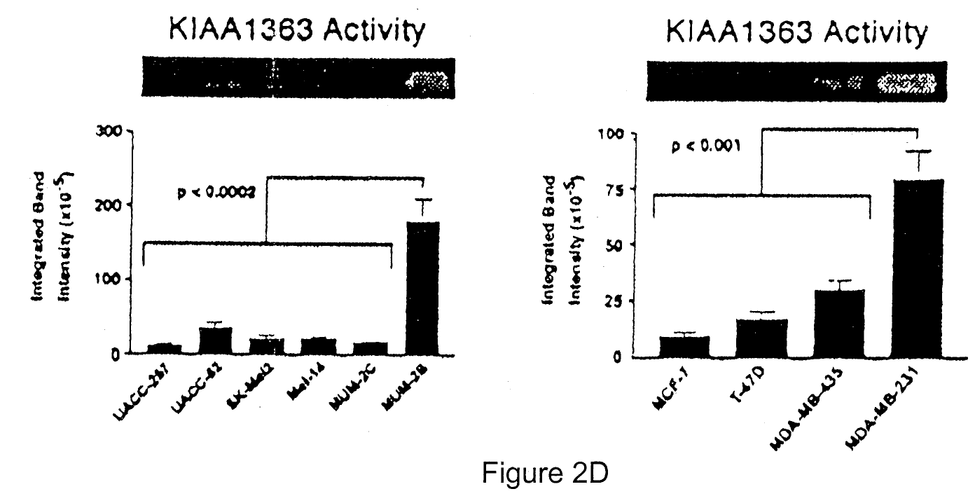

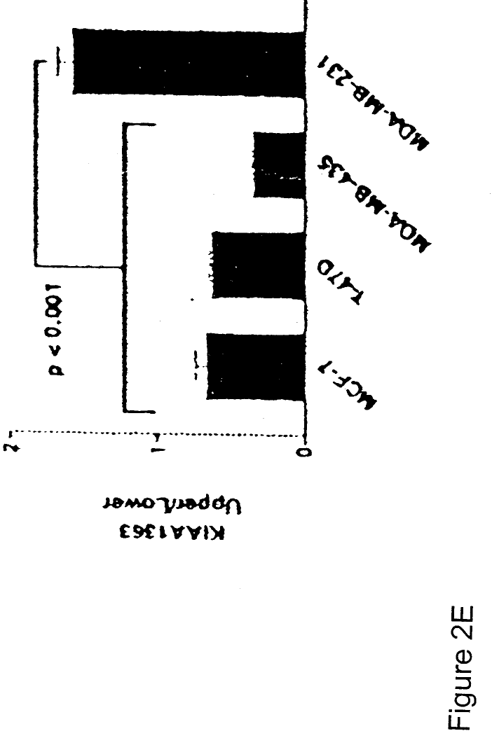

Collectively, these findings support the premise that protease activity in vivo is dictated by a balance between the levels of these enzymes and their endogenous inhibitors. Several membrane-associated serine hydrolase activities also exhibited restricted patterns of distribution across the cancer lines (FIG. 2A). Notably, the integral membrane enzyme, fatty acid amide hydrolase (FAAH) was detected exclusively in ER(+) breast carcinomas. The graded distribution of FAAH among breast cancer lines was used as a model to test the accuracy with which ABPP measured moderate as well as extreme differences in enzyme activity. FAAH activity was estimated by ABBP to be 2.5-fold higher in MCF7 cells relative to T-47D cells and a nearly identical ratio was calculated with assays using radiolabeled FAAH substrates (FAAHMCF7/FAAHT-47D =2.6;FIG. 2C). A second membrane serine hydrolase, the lysosomal enzyme palmitoyl-protein thioesterase 2 (PPT2) was upregulated in most melanoma lines relative to breast carcinomas. However, the ER(−) breast line MDA-MB-435 and the invasive melanoma line MUM-2B expressed high and low levels of PPT2 activity, respectively. Finally, a novel membrane-associated serine hydrolase activity KIAA1363 was upregulated in both invasive melanoma (MUM-2B) and breast carcinoma (MDA-MB-231) lines (FIG. 2D). Interestingly, this amidase was found to exist in two discrete glycosylation states that were themselves differentially expressed among the cancer lines. For example, the ratio of the upper to lower glycosylated forms of KIAA1363 was significantly higher in the MDA-MB-231 line relative to other breast cancer lines. (FIG. 2E).

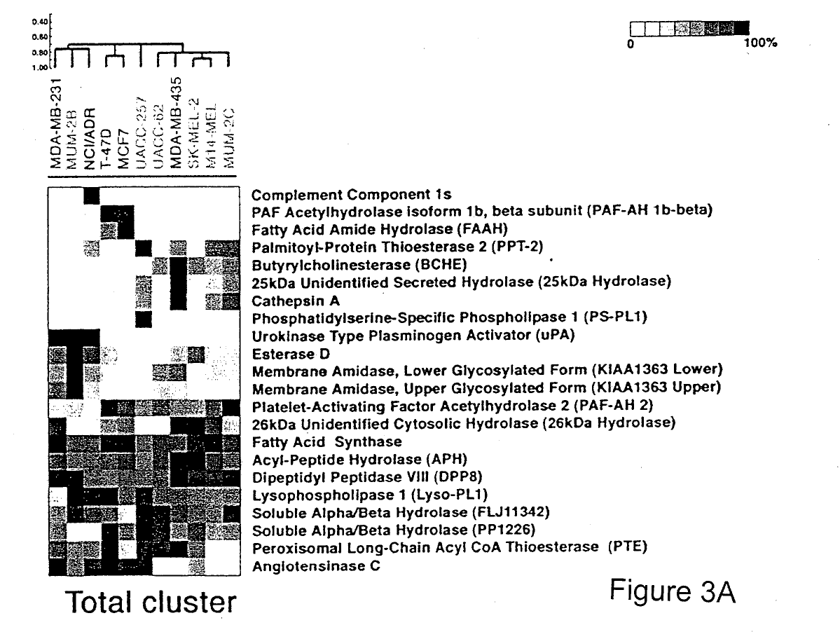

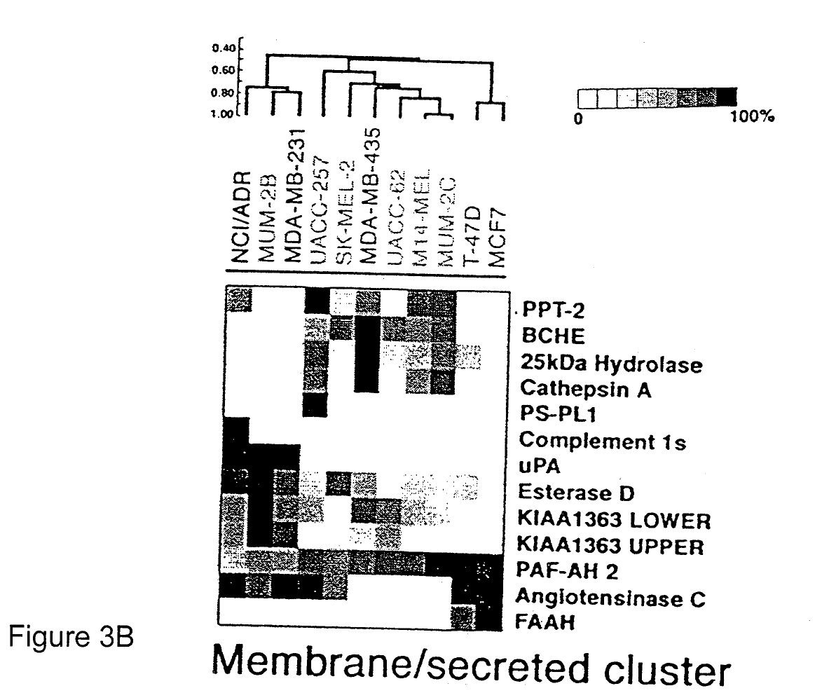

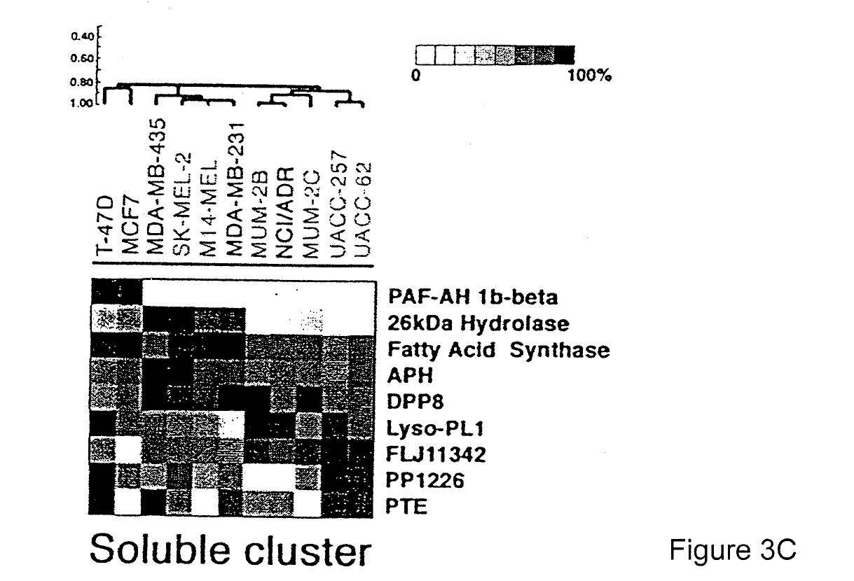

In contrast to the diverse patterns of enzyme activity observed in the secreted and membrane proteomes, the serine hydrolase activity profiles of the soluble proteomes of cancer cell lines appeared quite similar with few enzymes exhibiting restricted patterns of distribution (FIG. 2B). One notable exception was the enzyme activity PAF acetylhydrolase Ib,beta subunit, which was observed exclusively in ER(+) breast cancer lines. The serine hydrolase activity profiles of the secreted membrane, and soluble proteomes for each cancer cell line were merged and the resulting data sets analyzed with a hierarchical clustering algorithm and a pseudo-color visualization matrix (Eisen, et al., Proc. Natl. Acad. Sci. USA 1998, 95, 14863-68). (FIG. 3A).

Cancer cell lines were found to segregate into three major clusters that could be generally described as follows: a melanoma cluster (UACC-62,MDA-MB-435,SK-MEL-2, M14-MEL,MUM-2C), a breast carcinoma cluster (T-47D,MCF7), and an invasive cancer cluster (MDA-MB-231,MUM-2B,NCI/ADR). The ER(−) breast cancer line MDA-MB-435 was found as part of the melanoma cluster, providing proteomic support for the transcriptome-based hypothesis that this cell line may be melanoma in origin. This finding gains further significance when one considers that only six of the serine hydrolase activities described herein were represented on the cDNA microarrays originally used to classify MDA-MB-435 as a melanoma. Thus, despite only a modest overlap in the gene products analyzed, both ABPP and cDNA microarrays identified MDA-MB-435 as a potentially misclassified cell line. Although the majority of the cancer cell lines distributed into classes based on their tissues of origin, the most invasive breast carcinoma and melanoma lines (MDA-MB-231 and MUM-2B, respectively) formed a separate cluster that also included the aggressive adriamycin-resistant line NCI/ADR.

The features of the serine hydrolase activity profiles responsible for both origin-driven and phenotype-driven classifications were shown in additional cluster analyses in which enzyme activities from different subcellular fractions were separately examined. Nearly all of the enzymes that contributed to the observed classifications were found to reside in the secreted and membrane proteomes (FIG. 3B). In contrast, serine hydrolase activities from the soluble proteome mostly antagonized the observed classifications, possibly reflecting the domination of this proteomic fraction by broadly expressed “housekeeping” enzymes ((FIG. 3C). Several secreted/membrane enzymes were expressed selectively by either breast carcinomas (e.g., FAAH, angiotensinase C) or melanomas (e.g., BCHE, cathepsin A,PPT2), providing a driving force for the origin-based clusters (FIG. 3B and Table 1). However, the majority of these enzymes were strongly down regulated in the MDA-MB-231 and MUM-2B lines, which instead upregulated a distinct set of secreted and membrane enzyme activities (FIG. 3B and Table 1 ).Enzyme activities upregulated in these invasive lines included urokinase, a serine protease with a perceived role in tumorigenesis, and a novel membrane enzyme KIAA1363 for which no prior link to cancer had been made. Collectively, these results indicate that aggressive cancers share proteomic signatures that are more representative of their cellular phenotype than tissue of origin, supporting the conclusion that cancer progression may be accompanied by reversion to a pluripotent embryonic-like state.

It is evident from the above results that using probes that react with the active conformation of cells can be very informative as to a number of characteristics of the cells. By using probes that bind at catalytically active sites of enzymes, particularly where the probes are able to bind a multiplicity of members of a class of enzymes, one obtains a proteomic profile of the cells. This information may then be used in staging cancers, identifying targets for treatment, guiding the therapy, identifying the origin of the cells, and the like. By using cell lines and primary cells, one can develop a library of information that can be used as a prognosticator of outcome and method of treatment. One may also follow the results of the treatment, as changes in the proteomic profile. In employing probes that allow for easy determination of the reaction product of the probe and target proteins, the results are rapidly and efficiently determined and direct comparisons can be made between different samples. Cluster analysis of a body of data allows for rapid comparisons between samples and patients, providing valuable information to the health provider. The subject method also allows for the identification of proteins that are associated with particular characteristics of a cell, such as origin, aggressiveness, invasiveness, response to treatment, and the like. The subject invention provides a valuable resource in the armamentarium in the prevention and treatment of disease.

### EXAMPLE 2

### Enzyme Activity Profiles of the Secreted and Membrane Proteome that Depict Cancer Cell Invasiveness

By primarily measuring changes in transcript and protein abundance, conventional genomics and proteomics methods may fail to detect significant posttranslational events that regulate protein activity and, ultimately, cell behavior. To address these limitations, activity-based proteomic technologies that measure dynamics in protein function on a global scale would be of particular value. Here, we describe the application of a chemical proteomics strategy to quantitatively compare enzyme activities across a panel of human breast and melanoma cancer cell lines.

A global analysis of the activity, subcellular distribution, and glycosylation state for the serine hydrolase superfamily resulted in the identification of a cluster of proteases, lipases, and esterases that distinguished cancer lines based on tissue of origin. Strikingly, nearly all of these enzyme activities were down-regulated in the most invasive cancer lines examined, which instead up-regulated a distinct set of secreted and membrane-associated enzyme activities.

These invasiveness-associated enzymes included urokinase, a secreted serine protease with a recognized role in tumor progression, and a membrane-associated hydrolase KIAA1363, for which no previous link to cancer had been made. Collectively, these results suggest that invasive cancer cells share discrete proteomic signatures that are more reflective of their biological phenotype than cellular heritage, highlighting that a common set of enzymes may support the progression of tumors from a variety of origins and thus represent attractive targets for the diagnosis and treatment of cancer.

In recent years, DNA microarrays have become a standard tool for the molecular analysis of cancer, providing global profiles of transcription that reflect the origin (1-3), stage of development (4), and drug sensitivity (5) of tumor cells. The ability to complement these genomic approaches with methods that analyze the proteome (6, 7) is crucial for the identification and functional characterization of proteins that support tumorigenesis.

However, to date, the field of proteomics has had only a limited impact on cancer research, in large part because of the myriad technical challenges that accompany the analysis of complex protein samples (8). For example, conventional proteomics approaches that rely on two-dimensional gel electro-phoresis encounter difficulty analyzing important fractions of the proteome, including membrane-associated (9) and low abundance proteins (10). Additionally, most proteomics technologies are restricted to detecting changes in protein abundance (11), and therefore, offer only an indirect readout of dynamics in protein activity.

Numerous posttranslational forms of protein regulation, including those governed by protein-protein inter-actions, remain undetected. To address these limitations, we have developed a chemical proteomics strategy referred to as activity-based protein profiling (ABPP) that allows significant fractions of the enzyme proteome to be analyzed in an activity-dependent manner (12). This approach employs chemical probes that covalently label the active sites of enzyme superfamilies in a manner that provides a direct readout of changes in catalytic activity, distinguishing, for example, functional proteases from their inactive zymogens and or endogenously inhibited forms (12-14). Moreover, by providing a covalent link between the labeled proteins and a chemical tag, ABPP permits the consolidated detection, isolation, and identification of active enzymes directly from complex proteomes (13).

The present invention shows that ABPP probes that target the serine hydrolase superfamily of enzymes generate molecular profiles that classify human breast and melanoma cancer cell lines into subtypes based on higher-order cellular properties, including tissue of origin and state of invasiveness.

Materials and Methods

Preparation of Human Cancer Cell Line Proteomes.

All cell lines, with the exception of MUM-2B and MUM-2C, are part of the NCI60 panel of cancer cell lines and were obtained from the National Cancer Institute's Developmental Therapeutics Program. The MUM-2B and MUM-2C lines were provided by Mary Hendrix. All cell lines were grown to 80% confluence in RPMI medium 1640 containing 10% FCS and then cultured in serum-free media for 48 h, after which conditioned media was collected on ice and the cells were harvested.

Conditioned media samples were centrifuged at 2,400 g for 5 min, and the protein content of the supernatant was precipitated with ammonium sulfate (80%), resuspended in 50 mM Tris HCl, (pH 7.5; Buffer 1), and desalted over a PD-10 column (Amersham Pharmacia) to provide secreted proteome fractions. Cell pellets were sonicated and Dounce homogenized in Buffer 1 followed by centrifugation at 100,000× g to provide soluble cellular proteome fractions (supernatant) and a membrane pellet. Membrane pellets were homogenized in Buffer 1 with 1% Triton X-100, rotated at 4° C. for 1 h and then centrifuged at 100,000 g to provide membrane proteome fractions (supernatant). A typical ratio of 8:2:1 was observed for the relative quantity of soluble secreted membrane protein isolated for each cell line.

Proteome Labeling and Quantification of Enzyme Activities.

Standard conditions for fluorophosphonate (FP)-proteome reactions were as follows: proteomes were adjusted to a final protein concentration of 1 mg/ml in Buffer 1 and treated with 1 or 4 M (soluble membrane and conditioned medium proteomes, respectively) rhodamine-coupled FP (15) for 1 h at room temper-ature. After labeling, a portion of each proteome sample was treated with PNGaseF (New England Biolabs) to provide de-glycosylated proteomes.

Where indicated, proteome samples were preincubated with recombinant plasminogen activator inhibitor (PAI)-1 (20 g/ml; Calbiochem) for 30 min before the addition of FP-rhodamine. Reactions were quenched with one volume of standard 2 SDS PAGE loading buffer (reducing), separated by SDS PAGE (10-14% acrylamide), and visualized in-gel with a Hitachi FMBio IIe flatbed fluorescence scanner (MiraiBio) as described (15).

Integrated band intensities (normalized for volume) were calculated for the labeled proteins. For each enzyme activity, 4-6 data points were generated from independent labeling reactions conducted on 2 or 3 independently prepared proteomic samples. These data points were averaged to provide the level of each enzyme activity in each cell line. The activity levels of each enzyme were compared across the cell lines by using the Tukey's honestly significant difference test, where P values 0.05 were considered statistically significant.

Isolation and Identification of FP-Labeled Enzyme Activities.

Isolation of FP-labeled proteins was achieved by using biotinylated FPs and an avidin-based affinity purification procedure (13). Avidin-enriched FP-labeled proteins were separated by SDS PAGE, and the protein bands were excised and digested with trypsin. The resulting peptides were analyzed by a combination of matrix assisted laser desorption mass spectrometry (MS) (Voyager-Elite time-of-flight MS instrument, PerSeptive Biosystems, Framingham, Mass.) and microcapillary liquid chromatography-electrospray tandem MS [1100 HPLC (Agilent, Palo Alto, Calif.) combined with a Finnigan LCQ Deca MS (Thermo Finnigan, San Jose, Calif.)]. The MS data were used to search public databases to identify the FP-labeled proteins as described (13).

Fatty Acid Amide Hydrolase (FAAH) Enzyme Actvity Assays.

FAAH enzyme activity assays were conducted by using ¹⁴C-oleamide as a substrate as described (16), with the exception that the reactions were conducted at pH 8.0.

Cluster Analysis of Proteomic Profiles.

Averaged cell line values for each serine hydrolase activity were compared, with the line that expressed the highest level of this activity being defined as 100%. The rest of the cell lines were expressed as a percentage of this highest activity to normalize the data sets. We then applied a hierarchical clustering algorithm to the cell lines by average linkage clustering using the Pearson correlation coefficient as the measure of similarity (Gene Cluster computer package; ref. 17).

Additional cluster analyses were performed on enzyme activity profiles of the secreted, membrane, and soluble proteomes separately and in all of their respective pair-wise combinations. Of these six additional cluster analyses, only the “membrane secreted” analysis produced a dendrogram with increased distances among the three major clusters observed in the “total” serine hydrolase activity analysis.

Invasion Assays.

Cell invasiveness was assessed by using BIO-COAT matrigel invasion chambers (Becton-Dickinson) accord-ing to the protocol provided by the manufacturer. Briefly, 1.5×10⁵ cells were seeded into each chamber in serum-free condi-tions, and incubated for 16 h at 37° C., 5% CO₂ 95% air. Invading cells on the bottom surface of the membrane insert were fixed, stained with crystal violet, and counted. Results expressed as number of invading cells refers to average number of invading cells per 8 fields counted (n=3-4 for each cell line).

Results

Activity-Based Profiling of Human Cancer Cell Proteomes.

We have previously described the generation of affinity tagged FPs as prototype ABPP probes that target the serine hydrolase super-family of enzymes (12, 13). Considering that serine hydrolases represent one of the largest and most diverse classes of enzymes in the human proteome, composing approximately 1% of all predicted gene products (18, 19), we hypothesized that a comprehensive examination of their catalytic activities would yield proteomic information of sufficient quantity and quality to portray higher-order cellular properties.

To test this hypothesis, we selected a panel of human cancer cell lines for comparative analysis by ABPP based on the following criteria: (i) they exhibit a diverse range of well characterized cellular properties, including differences in hormone responsiveness, invasiveness, and metastatic potential; (ii) they represent multiple lines derived from at least two distinct types of cancer, and therefore permit the comparison of proteomic expression patterns both within and between cancer classes; and (iii) they have previously been analyzed with gene expression microarrays, and therefore allow for a comparison between proteomic data and trancriptional profiles (2, 3, 5).

To profile serine hydrolase activities in the context of their subcellular localization, proteomes from each cell line were separated into three fractions (secreted, membrane, and soluble) before treatment with a rhodamine-tagged FP probe (15). Fluorescently labeled proteins were then separated by SDS PAGE and visualized in-gel by using a flatbed laser-induced fluorescence scanner. Integrated band intensities for each identified enzyme activity were averaged from 4-6 proteomic samples to provide the results presented in FIGS. 1-4 (complete results are provided in bar graphs, which are published as supporting information on the PNAS web site, www.pnas.org). In parallel experiments, biotinylated FP probes were used to affinity isolate the active enzymes, which allowed for their molecular identification by mass spectrometry methods.

Serine Hydrolase Activity Profiles of the Secreted Proteomes of Human Breast and Melanoma Cancer Cells.

FIG. 1A shows a representative in-gel fluorescence analysis of the secreted serine hydrolase activity profiles of human cancer cell lines. Initial profiles revealed that several enzyme activities migrated as faint, diffuse bands, suggesting that they existed in a highly glycosylated state. Therefore, a portion of each FP-labeled proteome was deglycosylated before separation by SDS PAGE, resulting in a striking increase in the resolution of these proteins [for example, see sialic acid 9-O-acetylesterase (SAE); FIG. 1B].

Most of the secreted serine hydrolase activities exhibited a restricted pattern of distribution among the human cancer lines. For example, three secreted enzyme activities, SAE, butyrylcholinesterase, and cathepsin A, were up-regulated in most melanoma lines relative to breast carcinomas. Notably, however, the estrogen receptor negative [ER(−)] breast line MDA-MB-435 secreted high levels of these activities (FIG. 1A). Interestingly, a recent cDNA microarray analysis revealed that the transcriptional profile of the MDA-MB-435 line more closely resembled those of melanoma cells than breast carcinoma cells, suggesting that this line may be misclassified (2). Thus, several secreted serine hydrolase activities were identified that appeared to represent markers for cells of melanoma origin. It was therefore surprising to observe that all of these proteins were dramatically down-regulated in the highly invasive melanoma line, MUM-2B. Instead, MUM-2B cells secreted high levels of active urokinase and esterase D, two serine hydrolases that were also up-regulated in other aggressive lines examined, including the ER(−) breast carcinoma MDA-MB-231 and the multidrug-resistant NCI ADR line.

Identification of Posttranslationally Regulated Serine Proteases.

The identification of active urokinase in several invasive cancer lines was intriguing considering that this serine protease is an established marker of human cancer progression in vivo (20). However, a major open question regarding urokinase is the degree to which its mRNA and or protein levels in tumor samples reflect the state of activity of the protein. Urokinase activity is regulated by a host of posttranslational mechanisms including zymogen processing and interactions with multiple endogenous inhibitory proteins (PAI-1, PAI-2, maspin, myoepithelium-derived serine proteinase inhibitor), that also have perceived roles in tumorigenesis (21-25).

With these factors in mind, it is noteworthy that urokinase mRNA levels failed to directly correlate with urokinase activity in the cancer lines examined. Whereas approximately equal levels of active urokinase were observed in the NCI ADR, MDA-MB-231, and MUM-2B lines (FIG. 1C Left), 1.5- and 3-fold more urokinase transcript were observed in the latter two lines (FIG. 1C Right), respectively, suggesting that posttranscriptional events regulated urokinase activity in these cells.

To confirm that ABPP probes could detect shifts in the balance of proteases and their endogenous regulatory proteins, excess PAI-1 was applied to each secreted proteome before treatment with FP-rhodamine. The addition of PAI-1 blocked more than 85% of the observed urokinase activity in each of the cancer lines without affecting the activity of other proteases (FIG. 1D; PAI-1 inhibition of urokinase activity: MDA-MB-231, 95%±3%; NCI ADR, 88%±4%, MUM-2B, 98%±1%; n =3 or 4 per cell line). Collectively, these findings underscore the value of activity-based proteomics methodologies that can measure the functional outcome of posttranslational events that regulate enzyme activity in vivo.

Serine Hydrolase Activity Profiles of the Membrane and Soluble Proteomes of Human Cancer Cells

Several membrane-associated serine hydrolase activities also exhibited restricted patterns of distribution across the cancer lines (FIG. 2A). Notably, the integral membrane enzyme, FAAH, was detected exclusively in the poorly invasive breast cancer lines MCF7 and T-47D. The graded distribution of FAAH among breast cancer lines was used as a model to test the accuracy and sensitivity with which ABPP could measure moderate as well as extreme differences in enzyme activity.

FAAH activity was estimated by ABBP to be 2.5-fold higher in MCF7 cells relative to T-47D cells (FIG. 2B Left), and a nearly identical ratio was calculated with assays using the radiolabeled FAAH subtrate¹⁴C-oleamide (FAAH_{MCF7} FAAH_{T-47D}=2.6; FIG. 2B Right). Taking into account the k_{cat} of FAAH for oleamide (approximately 2 s −¹ at pH 8.0; ref. 26) and the amount of total membrane protein loaded in each gel lane (15 μg), we estimate that the fluorescent signal observed for FAAH in the T-47D membrane protein sample corresponded to approximately 600 pg of active protein, or less than 0.0005% of the total T-47D cell proteome.

This measure of FAAH protein matched closely the value calculated by comparing the T-47D FAAH signal to signals of a serial dilution of purified FAAH protein labeled to completion with FP-rhodamine (800 pg; data not shown). These findings highlight that ABPP methods can detect changes in enzyme activity with a level of accuracy and sensitivity compatible with profiling low abundance proteins in complex proteomes.

A second membrane-associated serine hydrolase activity, protein KIAA1363, displayed a cellular expression profile similar to that of uPA, being strongly up-regulated in both invasive melanoma (MUM-2B) and breast carcinoma (MDA-MB-23 1) lines. Interestingly, this enzyme was found to exist in two discrete glycosylation states that were themselves differentially expressed among the cancer lines. For example, the ratios of the upper to lower glycosylated forms of KIAA1363 were inversely related in the MDA-MB-231 and MDA-MB-435 lines (FIG. 2C).

In contrast to the diverse patterns of enzyme activity observed in the secreted and membrane proteomes of the cancer cell lines, the activity profiles of the soluble proteomes appeared quite similar, with few enzymes exhibiting restricted patterns of distribution.

Classification of Human Cancer Cells Based on Serine Hydrolase Activity Profiles

Serine hydrolase activity profiles of the secreted, membrane, and soluble proteomes for each cancer cell line were merged and the resulting data sets analyzed with a hierachical clustering algorithm and a psuedo-color visualization matrix (FIG. 3A) (17). Cancer cell lines were found to segregate into three major clusters that could be generally described as follows: a melanoma cluster (UACC-62, MDA-MB-435, SK-MEL-2, M14-MEL, MUM-2C), a breast carcinoma cluster (T-47D, MCF7), and an invasive cancer cluster (MDA-MB-231, MUM-2B, NCI ADR). Notably, the presence of the ER(−) breast cancer line MDA-MB-435 in the melanoma cluster provides the first proteomic support for the recent transcriptome-based hypothesis that this cell line may represent a misclassified melanoma line (2).

To understand the features of the enzyme activity profiles responsible for both origin-driven and phenotype-driven classifications, we performed additional cluster analyses in which enzyme activities from different subcellular fractions were separately examined. Strikingly, nearly all of the serine hydrolase activities that contributed to the observed classifications were found to reside in the secreted and membrane proteomes (FIG. 3B).

In contrast, serine hydrolase activities from the soluble proteome mostly antagonized the observed classifications, possibly reflecting the presence of many broadly expressed “house-keeping” enzymes in this proteomic fraction (FIG. 3C). Several secreted and membrane-associated enzyme activities were expressed selectively by either breast carcinomas (e.g., FAAH, angiotensinase C) or melanomas (e.g., butyrylcholinesterase, cathepsin A, PPT2, SAE), providing a driving force for the origin-based clusters (FIG. 3B). However, the majority of these enzymes activities were strongly down-regulated in the invasive melanoma and breast carcinoma lines, MUM-2B and MDA-MB-231, respectively, which instead up-regulated a distinct set of secreted and membrane-associated enzyme activities (FIG. 3B) that included urokinase, a serine protease with a well characterized association with tumorigenesis (20, 21), and the novel membrane-associated enzyme KIAA1363.

Characterization of a Membrane-Associated Serine Hydrolase KIAA1363 as a Marker for Cancer Cell Invasiveness.

The up-regulation of KIAA1363 in invasive cancer lines suggested that this enzyme may represent a new marker of tumor progression. Consistent with this notion, database searches revealed that the gene encoding KIAA1363 localizes to 3q26, a chromosomal region highly amplified in a variety of malignant cancers (27), including nearly 50% of advanced stage ovarian tumors (28). To further explore the relationship between KIAA1363 activity and cancer cell invasiveness, we determined the levels of activity of this enzyme across a panel of human ovarian cancer lines and correlated these values with measurements of invasiveness.

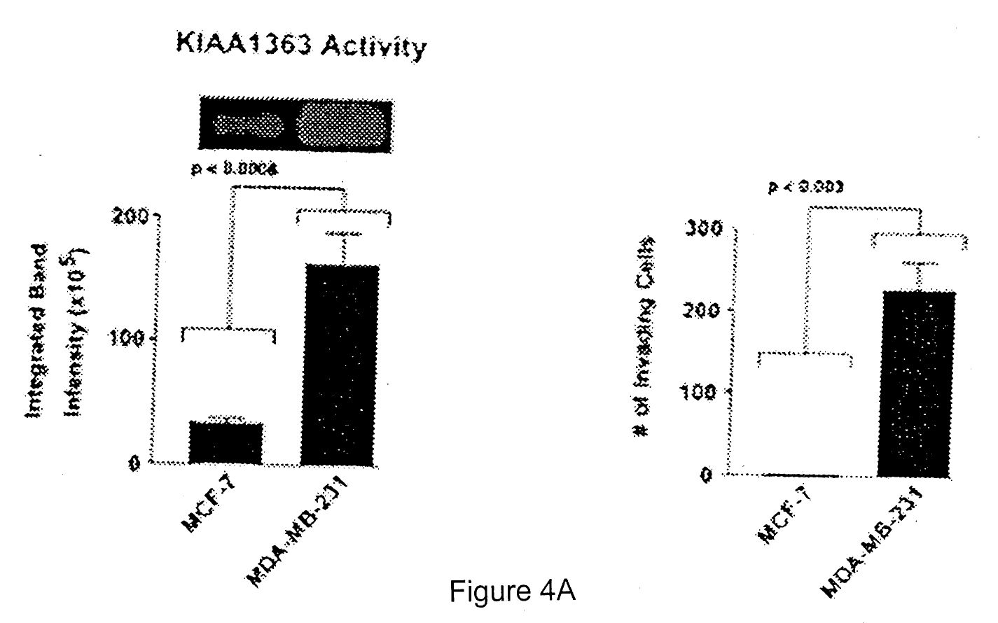

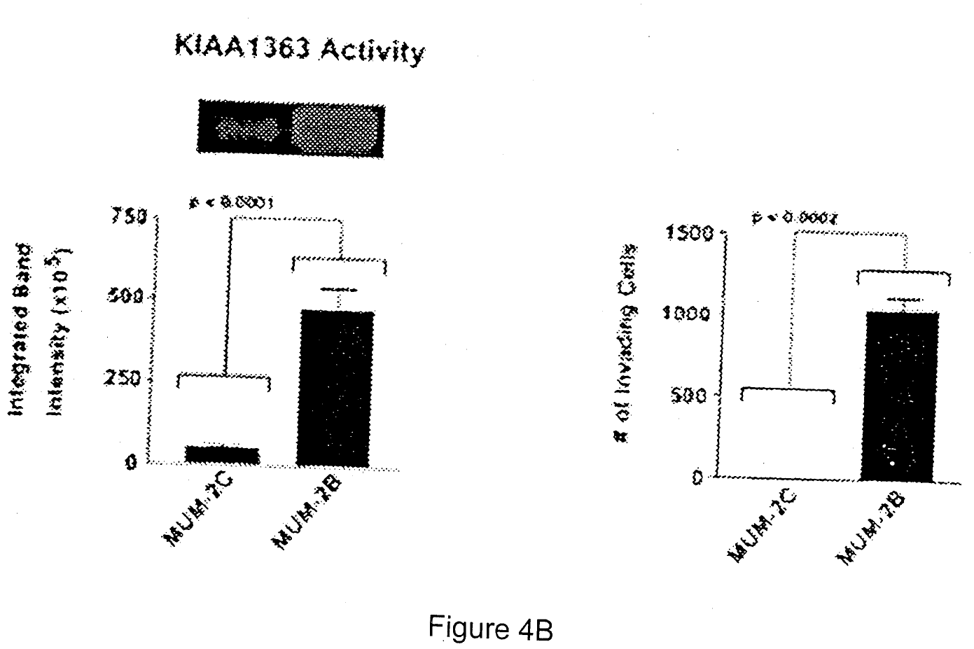

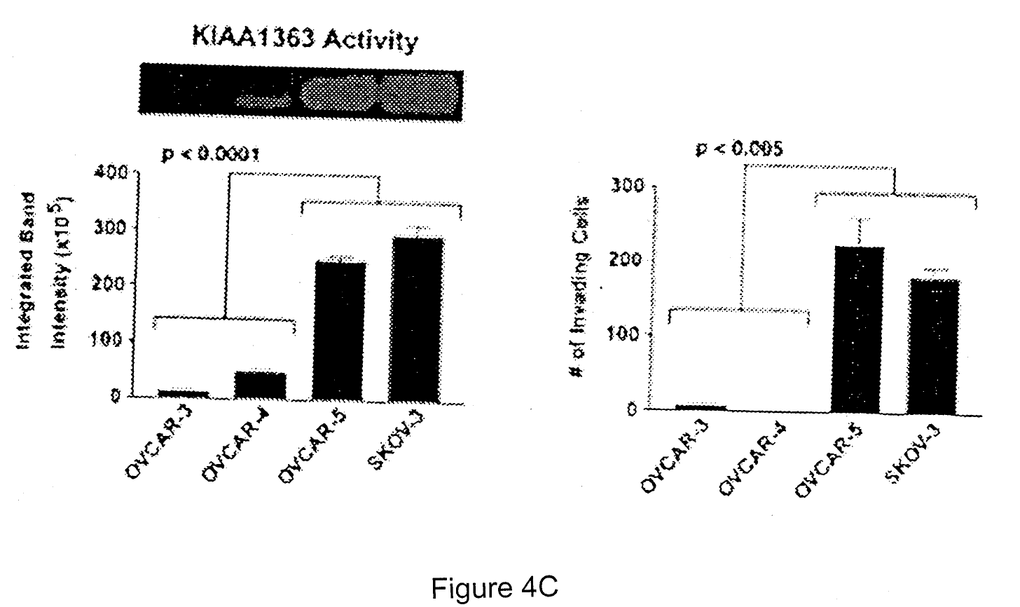

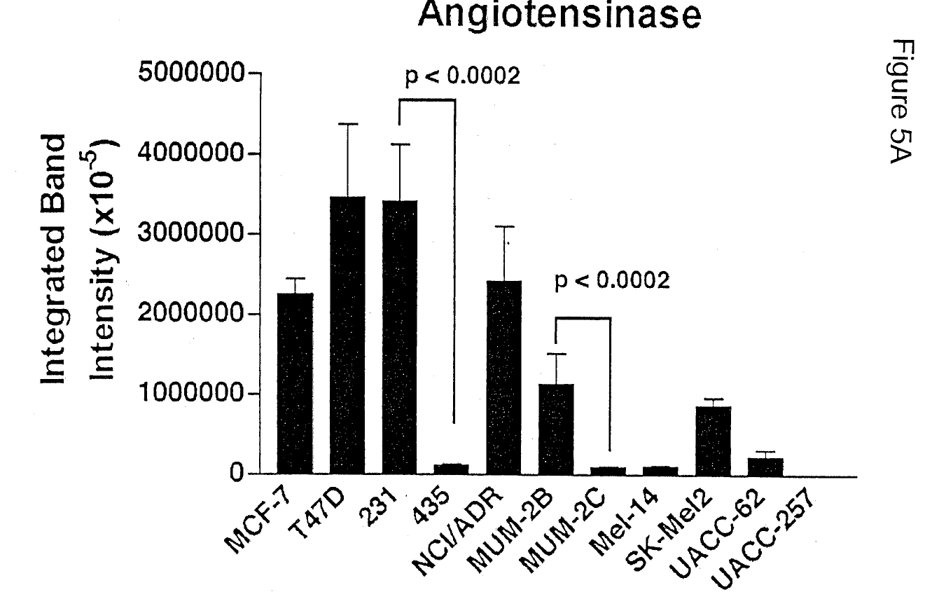

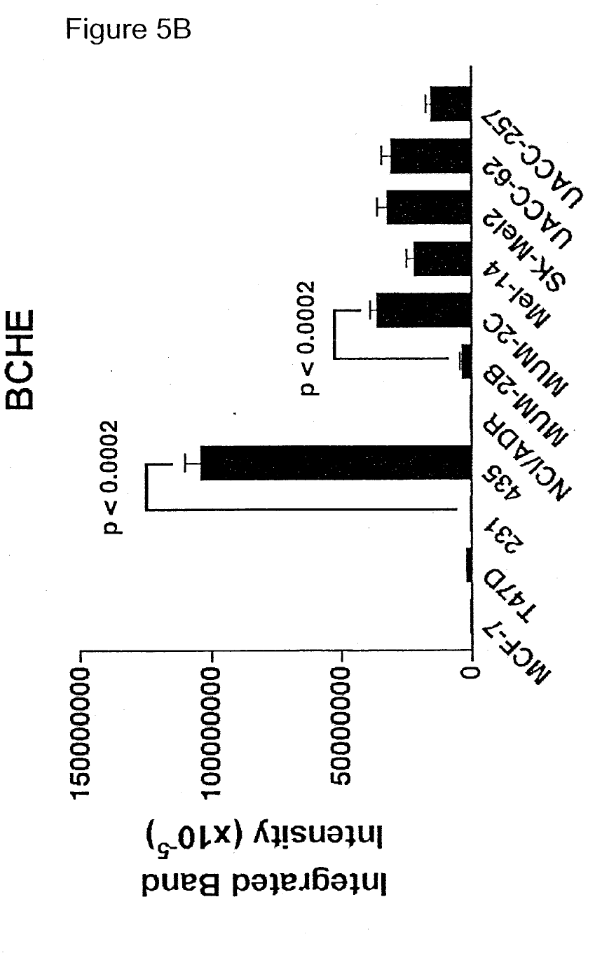

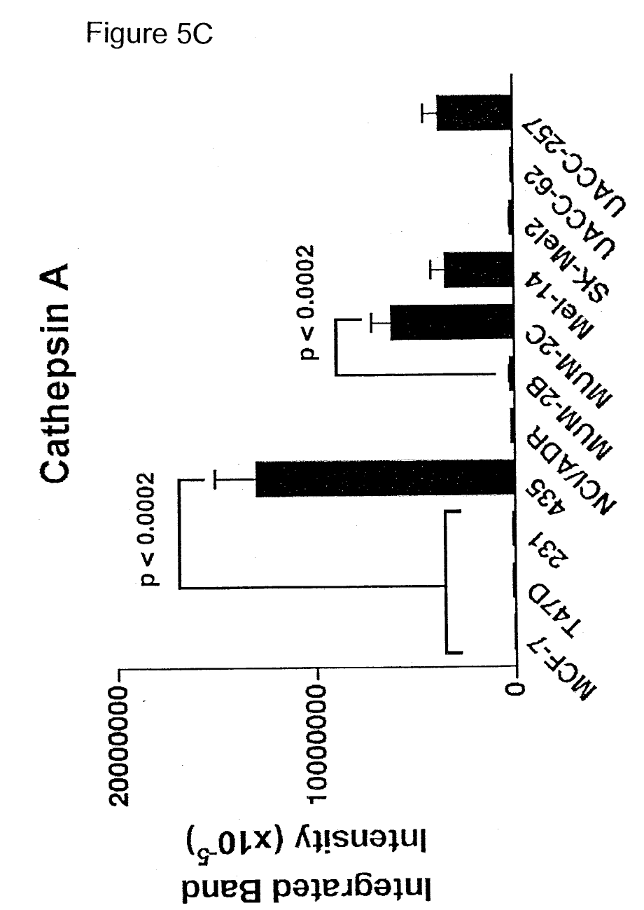

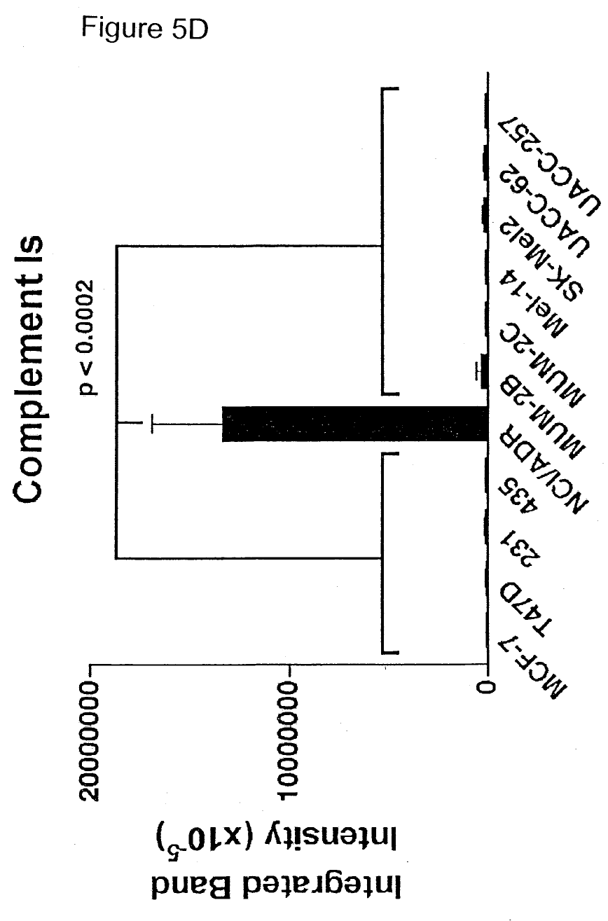

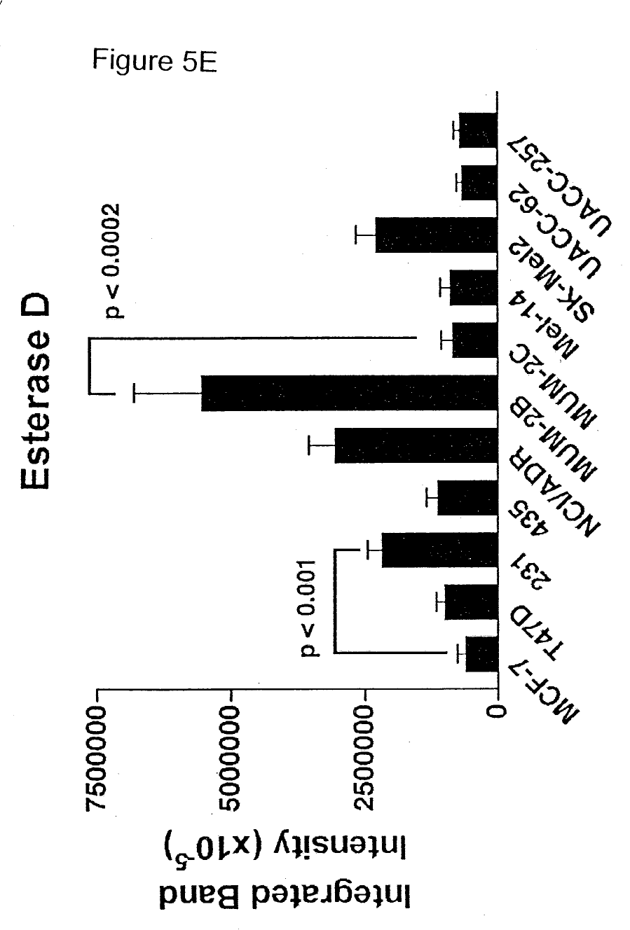

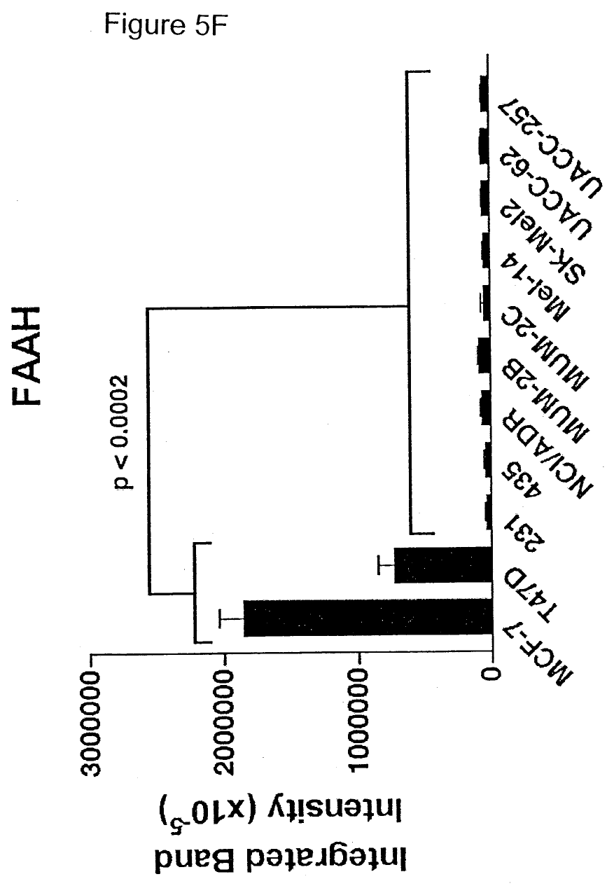

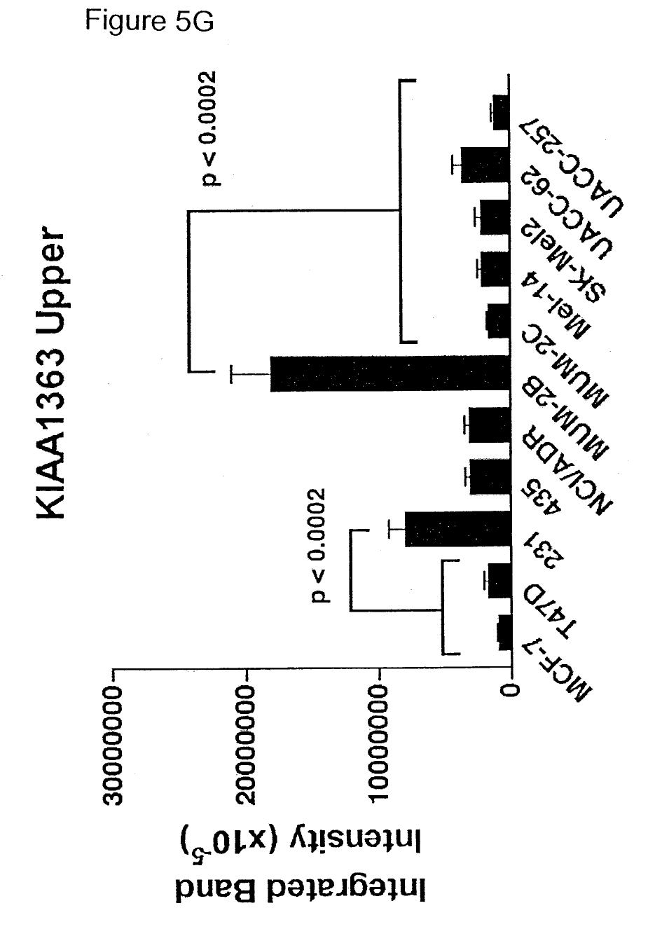

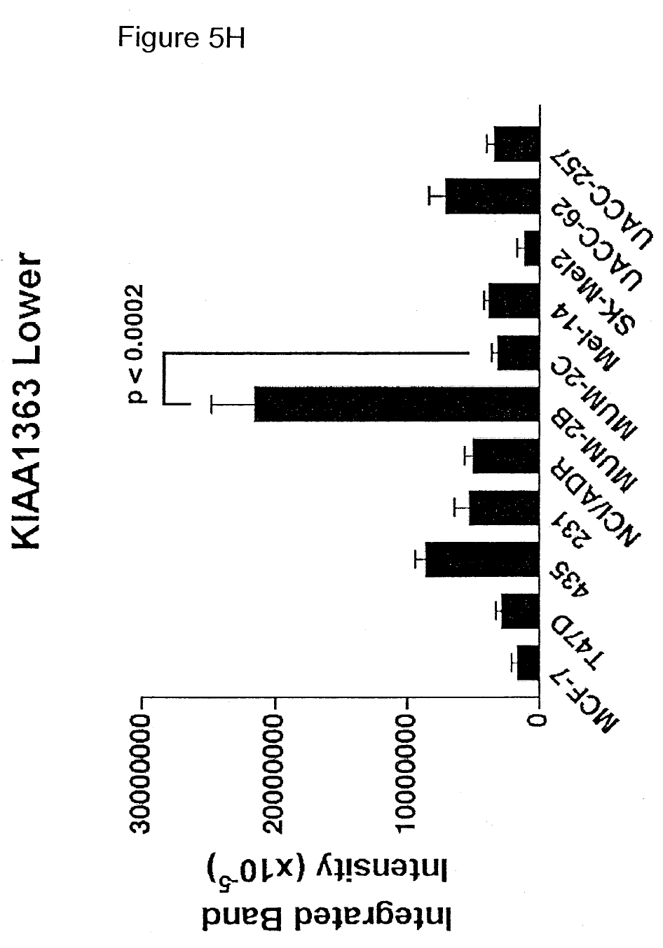

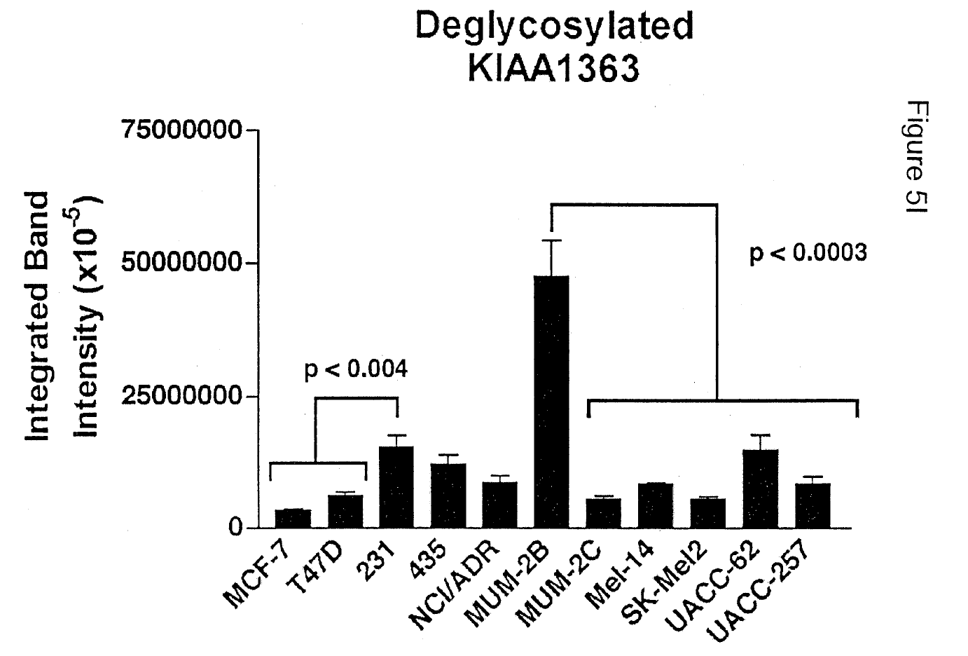

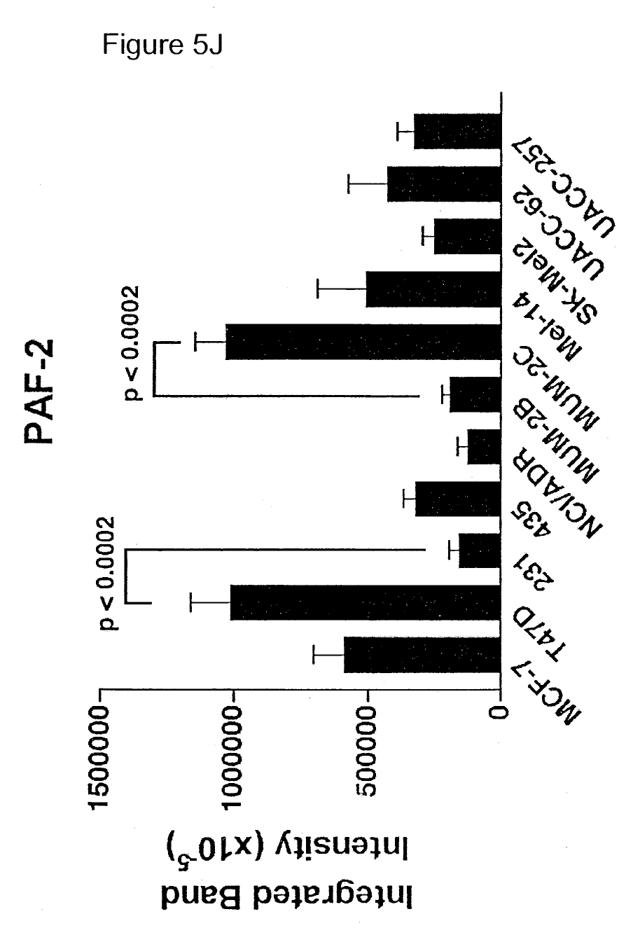

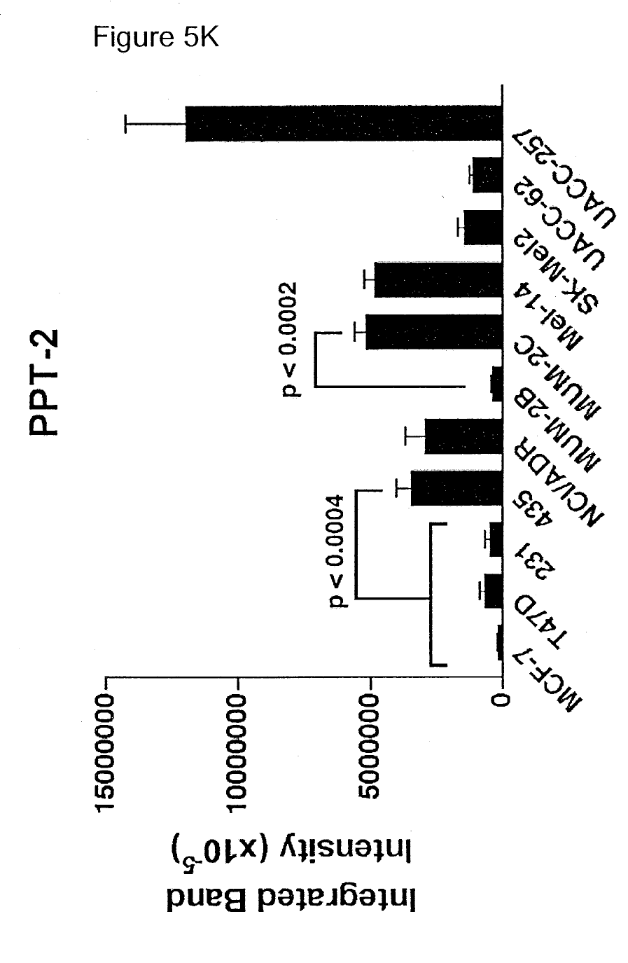

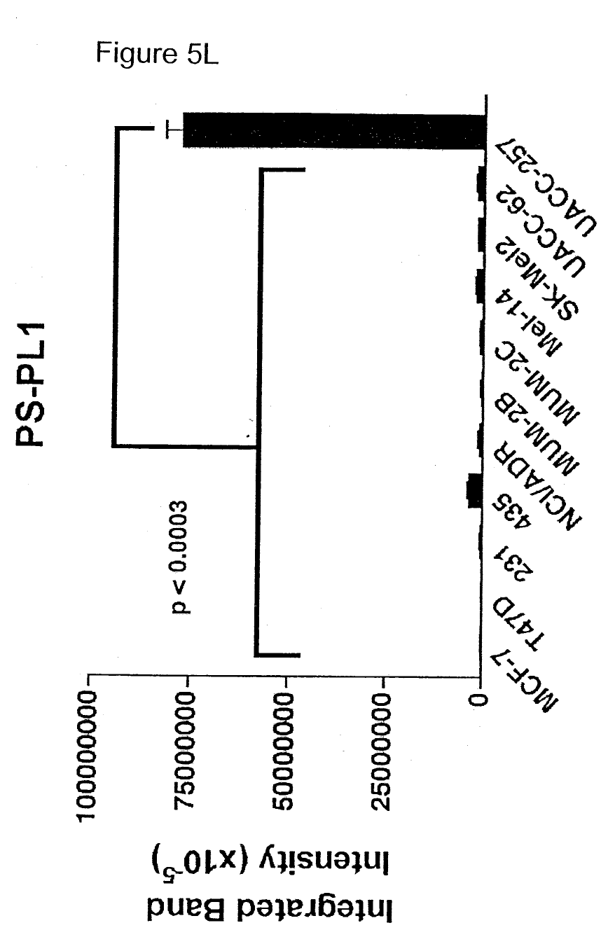

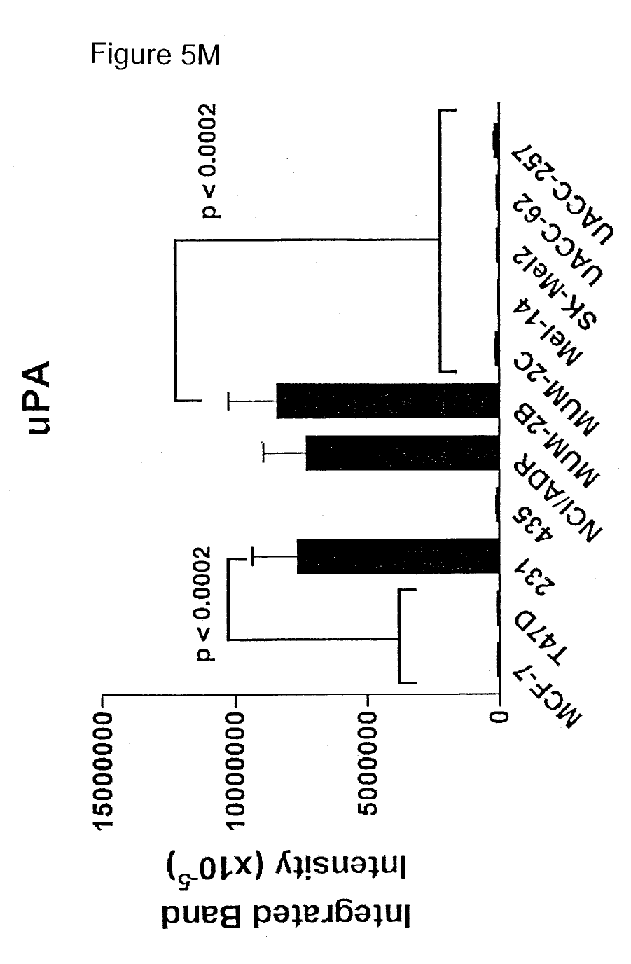

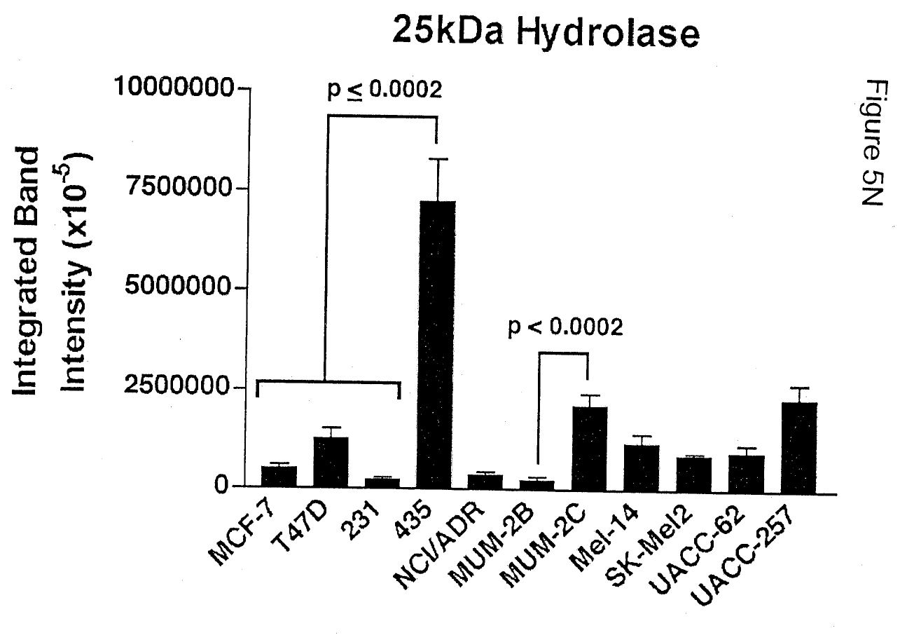

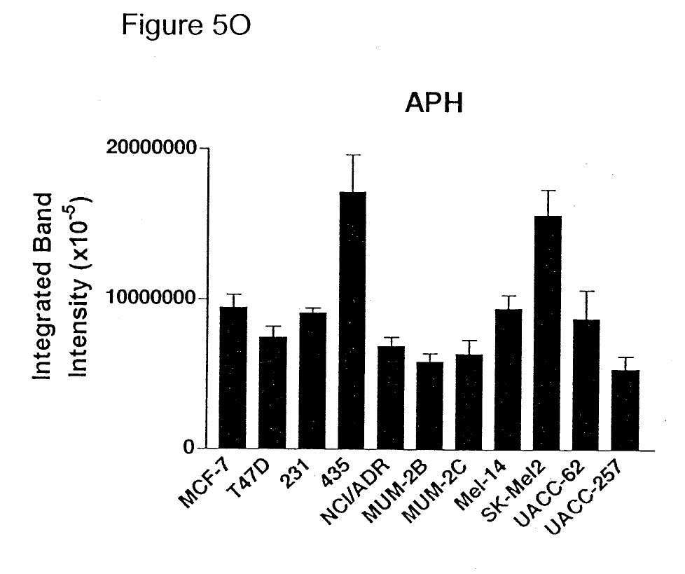

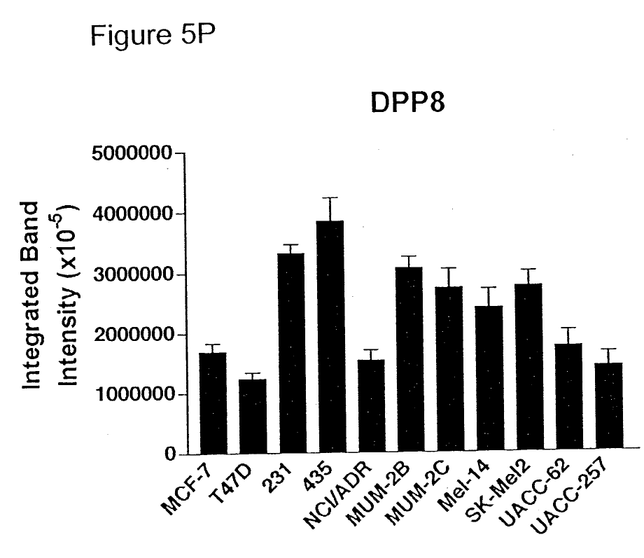

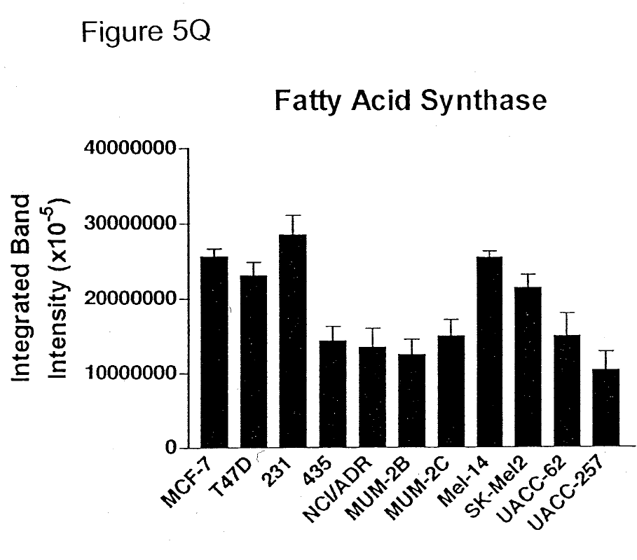

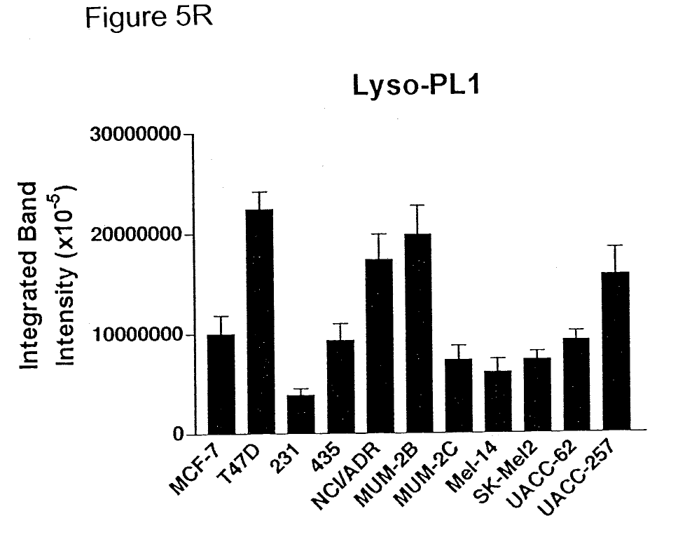

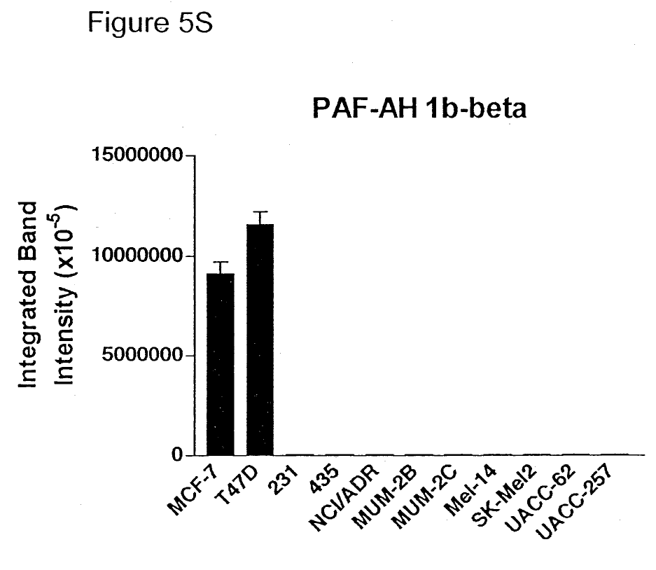

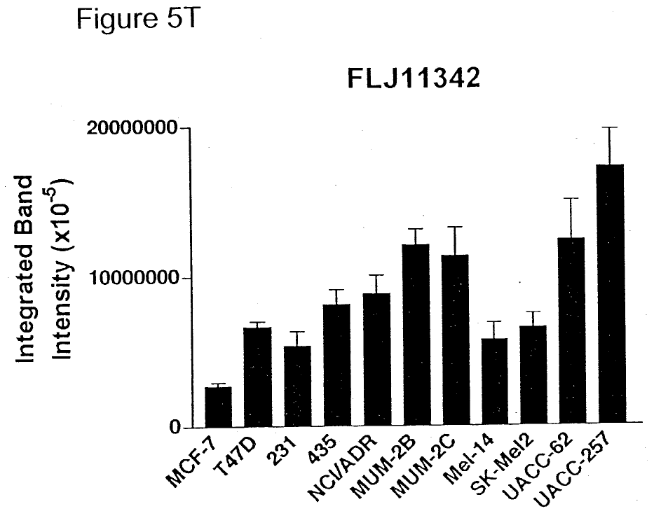

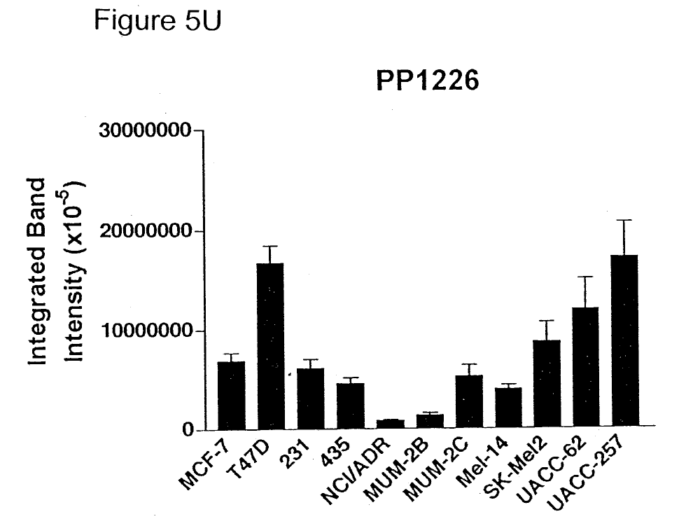

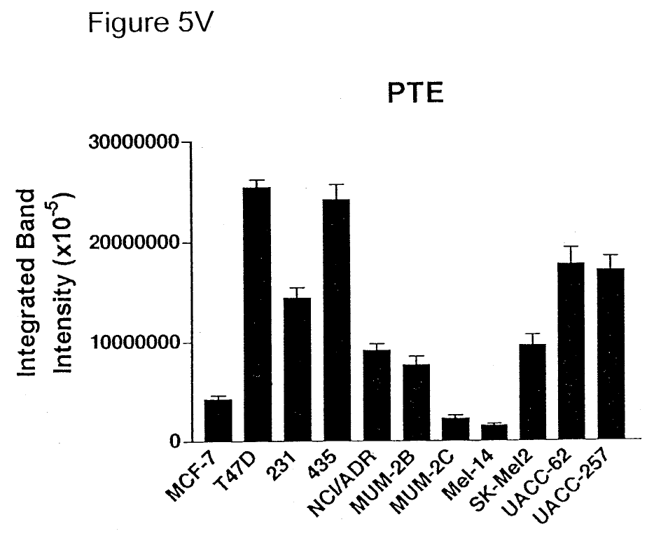

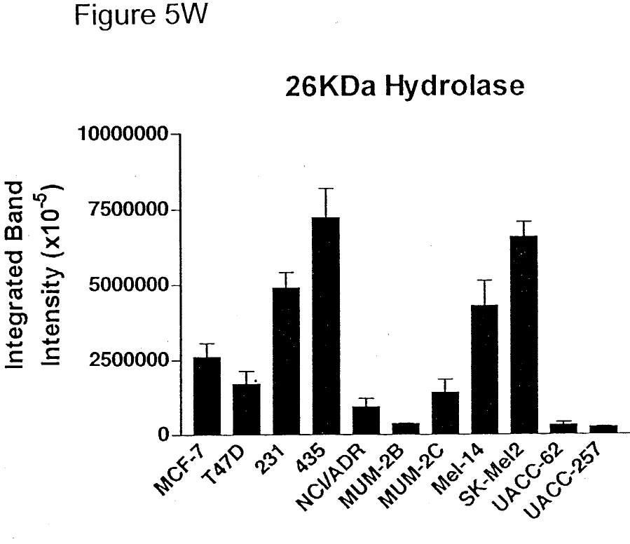

We selected for analysis a group of four ovarian carcinoma lines that, despite forming a discrete cluster based on global gene expression profiles (2), were otherwise relatively uncharacterized in terms of their cell biological properties, including invasiveness. The strong positive correlation that we observed between the levels of active KIAA1363 and cell invasiveness in breast carcinoma (FIG. 4A) and melanoma (FIG. 4B) lines directly extended to the ovarian carcinoma lines (FIG. 4C). Specifically, the two ovarian carcinoma lines that displayed high invasiveness (OVCAR-5, SKOV-3) were found to exhibit 5- to 25-fold higher levels of active KIAA1363 than the two noninvasive ovarian carcinoma lines (OVCAR-3, OVCAR-4). Thus, activity levels of the novel membrane-associated enzyme KIAA1363 correlated with pronounced differences in the invasiveness of cell lines derived from three distinct types of cancer, even in a case where this cellular phenotype was not reflected at the level of global gene expression profiles.

A proteome-wide analysis of variations in serine hydrolase activity permits the classification of human cancer lines into functional subtypes based on tissue of origin and state of invasiveness. Considering that most of the enzyme activities that contributed to the observed classifications resided in the secreted and membrane proteomes, these cellular fractions may contain molecular markers especially representative of differences in cancer cell behavior.

Furthermore, many of the secreted and membrane-associated enzymes were posttranslationally modified not only by glycosylation, but also by processing, as at least four enzymes, complement 1s protease, cathepsin A, urokinase, and SAE, were detected as single molecular species with masses 10-20 kDa lower than that predicted from their ORFs. The identification of SAE as an FP-reactive protein was particularly noteworthy given that this enzyme has not been classified in public databases as a serine hydrolase and shares no discernible sequence homology to any other functionally characterized protein.

The reactivity of SAE with FP-rhodamine, coupled with the enzyme's ability to hydrolyze esterified sialic acid groups (29), suggests that the protein is a member of the serine hydrolase superfamily. This finding indicates that even for intensively studied enzyme classes like the serine hydrolases, unrecognized members may exist in the human proteome that resist classification by primary sequence alignment.

The surprising finding that highly invasive cancer cells displayed secreted membrane serine hydrolase activity profiles nearly orthogonal to those displayed by their less aggressive counterparts suggests that invasive cancers share proteomic signatures that are more reflective of their cellular phenotype than tissue of origin. These results support that the advancement of cancers from a variety of origins may be accompanied by reversion to a common pluripotent embryonic-like state (30). Accordingly, enzyme activities, like KIAA1363, that are consistently up-regulated in invasive cancer lines derived from several different tumor types represent new bio-markers and or targets for the diagnosis and treatment of cancer. In general support of this notion, uPA, the enzyme activity that displayed the most similar profile to KIAA1363, is a well-established marker of tumor metastasis in vivo (20, 21) and a current target for multiple cancer drug development programs (31).

In summary, these studies highlight that proteomic approaches, like ABPP, that can analyze technically challenging fractions of the proteome (e.g., membrane, glycosylated, and low abundance proteins) are capable of generating molecular profiles that accurately depict higher-order cellular properties.

Finally, ABPP is a rapid and sensitive method for the comparative characterization of large numbers of proteomic samples, meaning that numerous cell types under a variety of experimental conditions can be analyzed in parallel, thereby accelerating the discovery of novel enzymes like KIAA1363, whose activities correlate with higher-order cellular properties. On this note, by using conventional two-dimensional gel electrophoresis proteomics technologies, a comparable analysis to the one described here would have required over 400 two-dimensional gels.

References

1. Golub, T. R., Slonim, D. K., Tamayo, P., Huard, C., Gaasenbeek, M., Mesirov, J. P., Coller, H., Loh, M. L., Downing, J. R., Caligiuri, M. A., et al.(1999) Science 286, 531-537.

2. Ross, D. T., Scherf, U., Eisen, M. B., Perou, C. M, Rees, C., Spellman, P., Iyer, V., Jeffrey, S. S., Van de Rijn, M., Waltham, M., et al.(2000) Nat. Genet. 24, 227-235.

3. Bittner, M., Meltzer, P., Chen, Y., Jiang, Y., Seftor, E., Hendrix, M., Radma-cher, M., Simon, R., Yakhini, Z., Ben-Dor, A., et al.(2000) Nature (London) 406, 536-540.

4. Perou, C. M., Sorlie, T., Eisen, M. B., van de Rijn, M., Jeffrey, S. S., Rees, C. A., Pollack, J. R., Ross, D. T., Johnsen, H., Akslen, L. A., et al.(2000) Nature (London) 406, 747-752.

5. Scherf, U., Ross, D. T., Waltham, M., Smith, L. H., Lee, J. K., Tanabe, L., Kohn, K. W., Reinhold, W. C., Myers, T. G., Andrews, D. T., et al.(2000) Nat. Genet. 24, 236-244.

6. Cravatt, B. F.& Sorensen, E. J.(2000) Curr. Opin.Chem. Biol. 4, 663-668.

7. Griffin, T. J.& Aebersold, R.(2001) J. Biol. Chem.276, 45497-45500.

8. Corthals, G. L., Wasinger, V. C., Hochstrasser, D. F.& Sanchez, J. C.(2000)

9. Santoni, V., Molloy, M.& Rabilloud, T.(2000) Electrophoresis 21, 1054-1070.

10. Gygi, S. P., Corthals, G. L., Zhang, Y., Rochon, Y.& Aebersold, R.(2000) Proc. Natl. Acad. Sci. USA 97, 9390-9395.

11. Anderson, N. L.& Anderson, N. G.(1998) Electrophoresis 19, 1853-1861.

12. Liu, Y., Patricelli, M. P.& Cravatt, B. F.(1999) Proc. Natl. Acad. Sci. USA 96, 14694-14699.

13. Kidd, D., Liu, Y.& Cravatt, B. F.(2001) Biochemistry 40, 4005-4015.

14. Adam, G. C., Cravatt, B. F.& Sorensen, E. J.(2001) Chem. Biol. 8, 81-95.

15. Patricelli, M. P., Giang, D. K., Stamp, L. M.& Burbaum, J. J.(2001) Proteomics 1, 1067-1071.

16. Cravatt, B. F., Giang, D. K., Mayfield, S. P., Boger, D. L., Lerner, R. A.& Gilula, N. B.(1996) Nature (London) 384, 83-87.

17. Eisen, M. B., Spellman, P. T., Brown, P. O.& Botstein, D.(1998) Proc. Natl. Acad. Sci. USA 95, 14863-14868.

18. Lander, E. S., Linton, L. M., Birren, B., Nusbaum, C., Zody, M. C., Baldwin, J., Devon, K., Dewar, K., Doyle, M., FitzHugh, W., et al.(2001) Nature (London) 409, 860-921.

19. Venter, J. C., Adams, M. D., Myers, E. W., Li, P. W., Mural, R. J., Sutton, G. G., Smith, H. O., Yandell, M., Evans, C. A., Holt, R. A., et al.(2001) Science 291, 1304-1351.

20. Konecny, G., Untch, M., Arboleda, J., Wilson, C., Kahlert, S., Boettcher, B., Felber, M., Beryt, M., Lude, S., Hepp, H., et al.(2001) Clin. Cancer Res.7, 2448-2457.

21. Bouchet, C., Spyratos, F., Martin, P. M., Hacene, K., Gentile, A.& Oglobine, J.(1994) Br.J.Cancer 69, 398-405.

22. Zou, Z., Anisowicz, A., Hendrix, M. J., Thor, A., Neveu, M., Sheng, S., Rafidi, K., Seftor, E.& Sager, R.(1994) Science 263, 526-529.

23. Xiao, G., Liu, Y. E., Gentz, R., Sang, Q. A., Ni, J., Goldberg, I. D.& Shi, Y. E.(1999) Proc. Natl. Acad. Sci. USA 96, 3700-3705.

24. DeClerck, Y. A., Imren, S., Montgomery, A. M., Mueller, B. M., Reisfel, R. A.& Laug, W. E.(1997) Adv.Exp.Med.Biol.425, 89-97.

25. Silverman, G. A., Bird, P. I., Carrell, R. W., Church, F. C., Coughlin, P. B., Gettins, P. G., Irving, J. A., Lomas, D. A., Luke, C. J., Moyer, R. W., et al.(1999) J.Biol.Chem.276, 33293-33296.

26. Patricelli, M. P., Lashuel, H. A., Giang, D. K., Kelly, J. W.& Cravatt, B. F.(1998) Biochemistry 37, 15177-15187.

27. Rooney, P. H., Murray, G. I., Stevenson, D. A., Haites, N. E., Cassidy, J.& McLeod, H. L.(1999) Br.J.Cancer 80, 862-873.

28. Sonoda, G., Palazzo, J., du Manoir, S., Godwin, A. K., Feder, M., Yakushiji, M.& Testa, J. R.(1997) Genes Chromosomes Cancer 20, 320-328.

29. Guimaraes, M. J., Bazan, J. F., Castagnola, J., Diaz, S., Copeland, N. G., Gilbert, D. J., Jenkins, N. A., Varki, A.& Zlotnik, A.(1996) J.Biol.Chem.271, 13697-13705.

30. Maniotis, A. J., Folberg, R., Hess, A., Seftor, E. A., Gardner, L. M., Pe'er, J., Trent, J. M., Meltzer, P. S.& Hendrix, M. J.(1999) Am.J.Pathol.155, 739-752.

31. Muehlenweg, B., Sperl, S., Magdolen, V., Schmitt, M.& Harbeck, N.(2001) Expert Opin.Biol.Ther.1, 683-691.

32. Greenbaum, D., Medzihradszky, K. F., Burlingame, A.& Bogyo, M.(2000) Chem. Biol. 7, 569-581.

33. Adam, G. C., Sorensen, E. J.& Cravatt, B. F.(2002) Nat.Biotechnol., in press.

34. Liu, et al., Proc. Natl. Acad. Sci. USA 96, 14694-99.

35. Kidd, et al., Biochemistry 40, 4005-15, 2000.

36. Adam, et al., Chem. Biol. 8, 81-95, 2001.

37. Scherf, et al., Nat. Genet. 24, 236-44, 2000.

38. Bittner, et al., Nature 406, 536-40, 2000.

39. Patricelli, et al., Proteomics 1, 1067-71, 2001.

Although the invention has been described with reference to the above examples, it will be understood that modifications and variations are encompassed within the spirit and scope of the invention. Accordingly, the invention is limited only by the following claims.

What is claimed is:

1. A method for evaluating cells in relation to their neoplastic state by determining the presence of a family of target enzymes having a common catalytic activity and having a common functionality in proximity to an active site associated with said common catalytic activity, said method comprising:
combining the protein components of said cells with at least one affinity based probe characterized by preferentially binding to an active site of a family of enzymes and comprising a detectable entity, a reactive functionality for bonding to a protein functionality in spatial proximity to said active site, and a linker, each probe characterized by comprising the same reactive functionality group specific for said target enzymes, whereby said probes react with any target enzymes present in said cell; and
determining the presence of the conjugate of each of said target enzymes with said probes by means of said detectable entity to obtain a profile of said target enzymes in said cell as said evaluation.

2. A method according to claim 1, wherein said profile is compared to profiles obtained with other cells.

3. A method according to claim 1, wherein said enzymes are serine/threonine hydrolases and said reactive functionality and recognition element comprise a fluorophosphonyl group.

4. A method according to claim 3, wherein said linker is an alkylene, oxyalkylene or poly(oxyalkylene) linker.

5. A method according to claim 1, wherein said determining comprises:
protease digestion of said conjugate to produce fragments; and mass spectrometric analysis of at least one of said fragments.

6. A method according to claim 1, wherein said evaluation is as to invasiveness, aggressiveness, hormone response or origin.

7. A method for evaluating cells in relation to their neoplastic state by determining the presence of a family of target serine/threonine hydrolases having a common functionality in proximity to an active site associated with a common catalytic activity, said method comprising:
combining the protein components of said cells with at least one affinity based probe characterized by: preferentially binding to an active site of said family of hydrolases, comprising a detectable entity consisting of a ligand or fluorescer, a fluorophosphonyl group, and a linker, whereby said probes react with any target hydrolases present in said cell; and
determining the presence of the conjugate of each of said target hydrolases with said probes by means of said detectable entity to obtain a profile of said target hydrolases in said cell as said evaluation.

8. A method according to claim 7, wherein said profile is compared to profiles obtained with other cells.

9. A method according to claim 7, wherein said linker is an alkylene, oxyalkylene or poly(oxyalkylene) linker.

10. A method according to claim 7, wherein said determining comprises:
protease digestion of said conjugate to produce fragments; and mass spectrometric analysis of at least one of said fragments.

11. A method according to claim 7, wherein said probe comprises a fluorescer and each of said conjugates is characterized by electrophoresis.

12. A method according to claim 7, wherein said target hydrolases comprise at least one of complement component 1s, PAF acetyl hydrolase isoform 1b, beta-subunit, FAAH, PPT-2, butyrylcholinesterase, p25, cathepsin A, PS_PL1, uPA, esterase D, KIAA 1363, PAF-AH 2, p26, fatty acid synthase, APH, DPP8, lysophospholipase 1, alpha/beta hydrolase, peroxisomal long-chain acyl CoA thioesterase or angiotensinase C.

13. A method according to claim 12, wherein said cells are melanoma cells.

14. A method according to claim 12, wherein said cells are breast epithelial cells.

15. A method for evaluating cells in relation to their neoplastic state by determining the presence of at least three of a family of target enzymes having a common catalytic activity and having a common functionality in proximity to an active site associated with said common catalytic activity, said method comprising:
identifying the presence in said cells as compared to a control cell of at least three members of the group consisting of complement component 1s, PAF acetyl hydrolase isoform 1b, beta-subunit, FAAH, PPT-2, butyrylcholinesterase, p25, cathepsin A, PS_PL1, active uPA, esterase D, KIAA 1363, PAF-AH 2, p26, fatty acid synthase, APH, DPP8, lysophospholipase 1, alpha/beta hydrolase, peroxisomal long-chain acyl CoA thioesterase and angiotensinase C,
wherein at least one of said three is FAAH, active uPA or KIAA 1363 whereby said neoplastic state is evaluated.

16. A composition comprising purified KIAA1363 characterized by being a serine/threonine hydrolase, present in melanoma and breast cancer cells, and comprising the amino acid sequence SEQ ID NO: 1.

17. A cluster analysis obtained from the method of evaluation according to claim 1.

18. A cluster analysis obtained from the method of evaluation according to claim 7.

19. An electrophoretic gel obtained by separating the conjugates obtained according to the method of claim 1.
